SEQUENCE LISTING (130 sequences)

SEQ ID NO: 1 - DNA, 20 bp, Artificial Sequence
  Antisense Oligonucleotide
tccgtcatcgctcctcaggg

SEQ ID NO: 2 - DNA, 20 bp, Artificial Sequence
  Antisense Oligonucleotide
gtgcgcgcgagcccgaaatc

SEQ ID NO: 3 - DNA, 20 bp, Artificial Sequence
  Antisense Oligonucleotide
atgcattctgcccccaagga

SEQ ID NO: 4 - DNA, 23865 bp, Homo sapiens
acaaaggacactgagacatttgttgctctatatcaaagaaaaaagtgtttgtcccaaaacttcaaaatgtgtaaattacacattctgcatctttacagctggagaaaattcactggcaatggaatatttaaaattagagcttgcttagtgtgctgcttctgatcactacttgatcccacttcgtgctttcatgttaattggcccaattggactctacagttggaaggtgaaaacttactatttcaacttgagtcacgtatgtattcttatcatatacttcttaaaggtactattttttttcttctgatagtcaccacaccaagcacttccagccaccctgccacagacttcctttgtaatcactgttgaaggacatgatgtttttatgacttcccgaaatgaaaaccctatcttgtttttaaaacaaacaaaccaacaaaaagtagtgtttatgtaagcattttgttccctgactctaggaacccctctgtttttatatcaactctgtactggcaaaacacaaaaacaaaatgccaccttgctaattcccttcctagcaaagtaatacagtttagcacatgttcaagaaaaaaatggctaagaaattttgtttccactaattattttcaagactgtgatatttacactctgctcttcaaacgttacattttataagactattttttaacatgttgaacataagccctaaatatatgtatccttaaattgtatttcaaatattttaggtcagtctttgctatcattccaggaatagaaagttttaacactggaaactgcaagtaaatatttgccctcttacctgaattttggtatccctctccccaagcttactttctgttgcagaaagtgtaaaaattattaaataaaattctaatgatggtatccgtgtggcttgcatctgatacagcagataaagaagttttatgaaaatggactcctgttccactgaaaagtaaatcttaatggcctgtatcaactatcctttgacaccatattgagcttgggaggaaggggaagtcctgaatgaggttataaagtaaaagaaaatatttgcaaaatgttcctttttttaaaatgttacattttagaaatattttaagtgttgtaacattgtaggaattaccccaataggactgattattccgcattgtaaaataagaaaaagttttgtgctgaagtgtgaccaggaagtctgaaaatgaagagagacagatgacaaaagaagatgcttctaatggactaaggaggtgctttcttaaagtcagaaagagatactcagaaagaggtacaggttttggaaggcacagagccccaacttttacggaagaaaagatttcatgaaaatagtgatattacattaaaagaagtactcgtatcctctgccactttatttcgacttccattgccctaggaaagagcctgtttgaaggcgggcccaaggagtgccgacagcagtctcctccctccaccttcttcctcattctctccccagcttgctgagccctttgctcccctggcgactgcctggacagtcagcaaggaattgtctcccagtgcattttgccctcctggctgccaactctggctgctaaagcggctgccacctgctgcagtctacacagcttcgggaagaggaaaggaacctcagaccttccagatcgcttcctctcgcaacaaactatttgtcgcaggtaagaaatatcattcctctttatttggaaagtcagccatggcaattagaggtaaataagctagaaagcaattgagaggaatataaaccatctagcatcactacgatgagcagtcagtatcaacataagaaatataagcaaagtcagagtagaatttttttcttttatcagatatgggagagtatcactttagaggagaggttctcaaactttttgctctcatgttccctttacactaagcacatcacatgttagcataagtaacatttttaattaaaaataactatgtacttttttaacaacaaaaaaaaagcataaagagtgacacttttttatttttacaagtgttttaactggtttaatagaagccatatagatctgctggattctcatctgctttgcattcagactattgcaatattgcacagaatgtagcctctggtaaactctgttgtacactcatgagagaatgagtgaaaaagacaaattacgtcttagaattattagaaatagctttcactttaggaactccctgagaattgctgctttagagtggtaaaataaataagcttctctttaaacggaatctcaagacagaatcagttacattaaaagcaaacaaaaaatttgcccatggttagtcatcttgtgaaatctgccacacctttggactgggctacaattggataatatagcattccccgagataattttctttcacaattaaggaaagggctgaataaatatctctgtttgaagttgaataacaaaaattaggaccccctaaattttaggactcctgaaattcgtctttttgcctatattcagctactttacgttctattaaatcttctttcaggccaggtgcactagctcatgcctagaatctcaggcaggcctgagcccaggaatttgagaccagccagggcaacacagtctctacaaaaaaataaaaaattacctgggtgtgttggtgcatgcctgtagaactactcaggatgctgaggactgcttgagcccaggatagccaaatctgtggtgagttcagccactaaacagagcgagactttctcaaaaaaacaaacaaaaaaacaaacaaacttccttcaaaataactttttatctgcaatgttttcctattgcctgtgagattaaatttactcttttacctgatttccaaagccctccataatctaatccgactttaccttgtgttcactgcaaaatagcaggactgttccactacaatccaaaaatcacaggttgggtgcagtggctcactcctgtaatcccaacactttggaaggccaaggcaggtggattgcttcagctcaggagttcaagaccagcctgggcaacatggcaaaaaccctgtctctccaaaacatacaaaaattagccagatgtggtagtatgtgcctgtagtcccaactactcaaaaggctaaggcaagaggatcacttgagcccaggaggtcaaggctacagtgagccatgtttgctgtgtcactgcactccagcctgggtgatagagcaagaccatgtctcaaaaaaaaaaaaaaaaagaaaagaaaagaaaaaaacatcgctctattcagttcacccccaccacaacattgttttgattatcacataaatgctggtccattgccttctctatctattcaaatctttaagcattctttgagattcaactcaattctccttttcaaactaggccatttaaactacatcagttccattttgatttttttgctttgagtctacagactcaaaaacaaaaacttaaaaacttattttttaagttttctgctactctcacctcttcaacactcacatacacgcattcataataagatggcagaatgttcaaggataaaatgatttatagaactgaaaagttaggttttgatcttgttgctgtcaagatgactacctacctgatctcaggtaattaattatgtagcatgctccctcatttcatcccatacctattcaacaggattggaattccacagcaaggataaacataatcatagttgcttttcaagttcaaggcattttaacttttaatctagtagtatgtttgttgttgttgttgttgtttgagatggagccctgctgtgtcacccaggctggagtgcagtggcacgaactcggctcactgcaacctctgcctcatgggttcaatcagttattctgcctcagtgtcccaagtagctgggactacaaggcacatgccaccatgcctggctaatttttgtatttttagtagaaacagggtttcaccatgttggccaggctggtctcgaactcctgacctcaagtgatccagccgcctcggcctcccaaagtgctggtattacaggcataagccaccgtgcccagcctaatagtatgtttttaaactcttagtggcttaacaatgctggttgtataataaatatgccataaatatttactgtcttagaattatgaagaagtggttactaggccgtttgccacatatcaatggttctctccttacagctttaactagagtctagaattgcaggttggtagagctggaacagaccttaaagactgactagccaacttccttgtccaaatgagggaactgagacccttaaaattaagtgacttgccccagacaaaactggaactcatgtgtcctaatttccatcatgaaattctaccattcactagcctctggctagttgtcaaagtattgcataactaaatttttatgtctgttttaaagaacaaattgtcactgcttactcctgggaggatctttctgaggtggtttataactcttaaaaaaaaaaaaagtcagtagtctgagaattttagatgaaatagtcaaagcatttttatccaatggatactataattttcatagattagagttaaagcaaagaaacacggatgagaaaggaagaggaaaattgaggagaggaggaatggggatgagaacacactacttgtaatcagtcatagatgtattgagaactaacaagaagaattgtaagaaaataagaatgaagaattcaaaatcaacacatgaaataaaaaagaaactactagggaaaaatggagaagacattagaaaaattattctatttttaaaattctgttttcaggcttccctcctgttcttcctccttctcattggctttcaggtggagggaaagtttaagatagaaaaaatatatatattctacacatccctttctacgctgttgtcatggcaacaaggtttatcatagcaaacttttattcatacaacatttattgagttcttactgtgtggtaagctctttccaggtgttgaaaattcaggggaaaaaagacaactcattgtcttaaaactcagatgaaagctgaacagacctatttttaatcaaagtaatctcaatttagggtagtaagagctatttaagaagcatgaacaggtgtgaaggaggtaggactctgaggagagaataattagctaggaatgaaagagcagagaagttttcctagaggaactattaaagctgggagttacgggatgaaagatgaggcagggtttgcaggcaaaaaaaaaaaaaaaggcaggggaaggggaagttctggcctggcagagagaataactgtggcaacaatggaggagagtctggaagcaagaaaaccaagtagaagagtattaaaatagaagatgccaggggtaatgagggcttgatttaaaacagtgctgttggagatggagaggagataccaaattctggagacatttctgagttagaatctacagtatttatcagacaagggaaagattagacaaaggagttaagaatgactcccaggtttcagtttggggcaggtaactaggacatgttttgaaaagtaatgtattagatctcttaccattggaactatgtatgtggagccaaattaaaatttgtacatgtatataactctccccccaccaccagtaactacttccctaactctctactttgtagccagacttcctaaaagaatagtttgtagtcactgtctttacttttcccctcccattctgtcctagatatttgtccacctaccatctgctgcctccactttacccaaactgttctacggttgcccaaaacttcctaattgccaaattcaatgaacaagtttaagcttatatgtaaattaggagctctacagtttgatttcgagcagcccctcctgaaaccctttctcttttgacttctgtgacacatctcagatttacaaaactgaactaattattttacacttgagctgtattttcgttcttttttcttaatgaatgaggtaaccactcaacaaattgcccaagccaaaaactacgaagtcatcctcagttcctccttcttctgtttgacccacaacagatcagctgagaaatcccgctgtttagtatgttaaaattacttcagtagttgttgtctgacctctgtccaatcttgttcaatcaggtccattcttttgttcttggtggtggtggtggtgttgacagagtttcgcttttgctgcccaggctgaagtgcagtggagcacttcactgcaaccacagcctcctgggtttaagcagttcaccctcccgagtagctgggactacaggtatgtgccaccacacccagctaattttgtgttttcagtagagacagggtttcaccatgttggtcaggctggtctcaaactcctgacctcaagcaatccacccacctcagcctcccaaagtgctgggattacaggcatgagccactgcacacggaccagatccattgtttatgttgcttctagagtgagtttttaaaacacaaatttgaccatatctttctcctatttaagtcagtattttttttttcaggaaaaaacagttcaaactctttagtctgcttacacaaggcctttgtagtctgactcttctttccaagctttcatcaaagtatactgcaagttgcattttatgtgaattgaattaggcaacggtataaaaattatagtttatatgggcaaaatggaaataatgttaactcttccaaatagtttatctagaatgacataatttcaaagctgtcaggtcaaatgagttataaactgttaacactattgccacatgcaagtgtctcttatacttggtagaattatctgcttccatgtcattattatgtaaattagactttaaataactcagaagttcttcagacatacaggttattattgtgctttttaaacataattttaaataattttatatatgataatgttatccaagtgctaagggatgtattgttactgctgtgcaaaaaaaaaaaaaaaaaaaaaaactccaaataaatatgttgaaaccaagtttatatgcaagaaaacaatattaaaaaggccaaagtaccaccataataggctgtgtggagacagcaggctacaaaacactagtaataatgctgagaaagttgaaaaaagaaagaaagcaacaatatgctttggttgttgtaggtttatgtactccaagaatatctcctctcaaacttttacgttttttccaaagaaaagttaactttggctgggcgcagtggctcttgcctgtagtcccagcctttgggaggccaaggcgggcagatcacctgaggtcaggagtttgagaccagcctgaccaacatggagaaacccgcccccctcactactaaaagaatacaaaattaggccgggcacagtggcttacccctgtaatcccagcactttgggaggccgaagcaggaagatcacctgaggtcaggagttcgagaccagccatggagaaacccgtctctactaaaaatacaaaattagccgggcgtggtggtgcatgactgtaatcccagctactcaggaggctaaggcagagaatcacttgaacccaggcagtggaggttgcagtgagccgagatcgtgccattgcactccagcctgggcaacaagagcgaaactctgtatccaaaaaacaaaagaaaagaaaaggtaaccttgaactatgtgagatctttagaaatgcattctttctgtaaaatgtgactacatttgccttatttatggtaaaaatgttgaggcctcaaacaacccatattttctcggtctccccgctgcctagcctttgttcacattgcttcttcttggtggaagctcttcctctggccttgaaaatgcctgcttctctttcaaggtagcacagtcatcactttctgtggtaaccttctccagcaccatcaaacagaaagaatgaatctcttgtaaattcagctcttacgtcattcattacattattttgtaactctttatagattcttctctcccactagactctgagtcactggagagtaggagccaactctcattcatgtgtggtttggtcagctactggccacattcctgatgcatagttaatgctcaaaccttaactggtgaatcagctcaaatattgtccttctctaaatccattcactcattgactaactatgtactcaaaatagtaaacaccagtaatttaatccaattcctgcccatactgcttggtacatttcaggtgaattagtttgataaatatgtgtgtattacataatattaaagtatgtacagaagatcatgctaatcattatttcaccaactgataactaatcaaaacatagagtgcctctcaggttaacaaatgtctgccttctcagttaatgcagtcattaacaaacaccttctgatgctgataatagggccttgttcagcaatgaagccataaaggtgaataaagaacatgccctcgtggagctcacagcctagtcattattgttctgatttttaatattaatgttggtttgggttttggtgaaaaatgtttagacttatcttagtgatcttttcatcctttgctatattatttttctctaagagtcttccttatcccctcctttaaaaaactaggtgataattctaaattgtaaatttaaatattataaatagcttataaaatttaatatttataatatttaaatgtttgataaatatttaaattttataatatttaaatgtttatttaaattcatttgtacatcagtttttattttatttaaatgtgttggccaggcatggtggctgacacctataatcccagaactttgagaggccaagtcaggcaaaccatttgagctcaggagtttgagaccaccctgggcaacgtggtgaaaccctgtctctaccaaacatatgaaaacttatctgggtgtggtggcacgcatctgtggtcccagatgggagtcccaggctaagatgggagaatcgcttgaacccaggtgagaggggtggggtggatgttgcagtgagctgagatcgtgccactgcactccaacctgggtgacagagtgagactccatctcaaaaaaaaaaaatgttatctaaataagataaatttaataactgttcgcacttagatgagcataaggaactaaacctagataaaactatcaaataaggcctgggtacagtgactcatgcctgtaatctcaagcactttgggaggccaaaattatacaaagttagttgtataacaccaactaacaactattttggggttagcttaattcagattaattttttttaaactgagttttaaattcctgcttactctaccatacatgctaggcctcatattatgctagaaaaattttgagcacagatttatgaatactctcctgcataccatttaatttttaaacaaattttaatgcagtatatatgtgcctttttaccaacacattaaataataagatctactgtgaggactaaacttctgtaatttcaaagtagtaatgagtttaaaccatgtctcaagatctctgcaataactgtagcacaacagaaaataggtatttctattaatgacagagtcacaagtactactaataatactgtggtttgtttcctgcaactaatcatgggaggaatgctaaatttcagaggttggtgaaaatacatgtgtatttttttccccatccaagttcacagatttctcacactgagaactcctattccataacaaaattctggaagcctgcacaccgtattggaagaggggcagaaaggaaaagcaaatggaaggatttaaatttttttcaaatcctgtatcccttgattttacagcaagattgtatttatgtattacttgtgttaaaaatatagtataatcgagactccagatcaaaaatcaccgcagctcagggagaaagagggccaccaaatgccagagcccttcagccttctcccaccctgcctgtaccctcagatggaagcacttttttatcattgtttcacctttagcattttgacaatgaagtcacaaaccttcagcctctcacccataggaacccactggttgtaagagaaggatgaagccagtccttcctaaagggcacgattagatgtgtttatggcatcctcaggtgaaactatatttatattgacaatatatttatatttctcaaggaatactagaataatgattcagttcagtactaggccatttatctaccctttataatattgtttaatgagaaaatgctttctatcttccaaatatctgatgatttgtaagagaacacttaaacatgggtattcataagctgaaacttctgggatttattgaatgtcaagattgttcatcagtatactaggtgattaactgaccactgaacttgaaggtagtataaagtagtagtaaaaggtacaatcattgtctcttaacagatggctctttgctttcattaggaataaagatggctgctgaaccagtagaagacaattgcatcaactttgtggcaatgaaatttattgacaatacgctttactttataggtaaggctaatgccatagaacaaataccaggttcagataaatctattcaattagaaaagatgttgtgaggtgaactattaagtgactctttttgtcaccaaatttcactgtaatattaatggctcttaaaaaaatagtggacctctagaaattaaccacaacatgtccaaggtctcagcaccttgtcacaccacgtgtcctggcactttaatcagcagtagctcactctccagttggcagtaagtgcacatcatgaaaatcccagttttcatgggaaaatcccagttttcattggatttccatgggaaaaatccccagtacaaaactgggtgcattcaggaaatacaatttcccaaagcaaattggcaaattatgtaagagattctctaaatttagagttctgtgaattacaccattttatgtaaatatgtttgacaagtaaaaattgattcttttttttttttttctgttgcccaggctggagtgcagtggcacaatctctgctcactgcaacctccacctcctgggttcaagcaattctcctgcctcagccttctgagtagctgggactacaggtgcatcccaccatgcctggctaattttttgtatttttactagagacagggttttgccatgttgtccaggctggtcttgaactcctgatctcagatgatcctcctgcctcggcctcccaaagtgctgggattacaggcatgaaccaccacacatggcctaaaaattgattcttatgattaatcttctgtgaacaatttggcttcatttgaaagtttgccttcatttgaaaccttcatttaaaagcctgagcaacaaagtgagaccccatctctacaaaaaactgcaaaatatcctgtggacacctcctaccttctttggaggctgaagcaggaggatcacttgagcctaggaatttgagcctgcagtgagctatgatcccacccctacactccagcctgcatgacagtagaccctgacacacacacacaaaaaaaaaccttcataaaaaattattagttgacttttcttaggtgactttccgtttaagcaataaatttaaaagtaaaatctctaattttataaaatttatttttagttacatattgaaatttttaaaccctaggtttaagttttatgtctaaattacctgagaacacactaagtctgataagcttcattttatgggccttttggatgattatataatattctgatgaaagccaagacagacccttaaaccataaaaataggagttcgagaaagaggagtagcaaaagtaaaagctagaatgagattgaattctgagtcgaaatacaaaattttacatattctgtttctctctttttccccctcttagctgaagatgatggtaaagtagaaatgaatttatttttctttgcaaactaagtatctgcttgagacacatctatctcaccattgtcagctgaggaaaaaaaaaaatggttctcatgctaccaatctgccttcaaagaaatgtggactcagtagcacagctttggaatgaagatgatcataagagatacaaagaagaacctctagcaaaagatgcttctctatgccttaaaaaattctccagctcttagaatctacaaaatagactttgcctgtttcataggtcctaagattagcatgaagtcatggattctgttgtagggggagcgttgcataggaaaaagggattgaagcattagaattgtccaaaatcagtaacacctcctctcagaaatgctttgggaagaagcctggaaggttccgggttggtggtggggtggggcagaaaattctggaagtagaggagataggaatgggtggggcaagaagaccacattcagaggccaaaagctgaaagaaaccatggcatttatgatgaattcagggtaattcagaatggaagtagagtaggagtaggagactggtgagagaagctagagtgataaacagggtgtagagcaagacgttctctcaccccaagatgtgaaatttggactttatcttggagataatagggttaattaagcacaatatgtattagctagggtaaagattagtttgttgtaacaaagacatccaaagatacagtagctgaataagatagagaatttttctctcaaagaaagtctaagtaggcagctcagaagtagtatggctggaagcaacctgatgatattgggacccccaactttcttcagtcttgtacccatcatcccctagttgttgatctcactcacatagttgaaaatcatcatacttcctgggttcatatcccagttatcaagaaagggtcaagagaagtcaggctcattcctttcaaagactctaattggaagttaaacacatcaatccccctcatattccattgactagaatttaatcacatggccacaccaagtgcaaggaaatctggaaaatataatctttattccaggtagccatatgactctttaaaattcagaaataatatatttttaaaatatcattctggctttggtataaagaattgatggtgtggggtgaggaggacaaaattaagggttgagaggctattattttagttattacaagaaatgatggtgtcatgaattaaggtagacataggggagtgctgatgaggagctgtgaatggattttagaaacacttgagagaatcaataggacatgatttagggttggatttggaaaggagaagaaagtagaaaagatgatgcctacatttttcacttaggcaatttgtaccattcagtgaaataggaaacacaggaggaagagcaggttttggtgtatacaaagaggaggatggatgacgcatttcgttttggatctgagatgtctgtggaacgtcctagtggagatgtccaaaaactcttctacatgtggttctgagttcaggacaaagatttgggctggagatagagatattgaaggcttatgcatagaaatggcatttgaatctatagagataaaaagacaaatcagaggaaatgtgtaaagtgagagaggaaaagccaagtactgaactggggggaatacctacatttaaaggatgcagtagaaagaagctaataaacaacagagagcagactaaccaaaagggaagaagaaaaaccaagagaattccaccgactcccaggagagcatttcaagattgagggaattccaccgactcccaggagagcatttcaagattgaggggataggtgttgtgttgaattttgcagccttgagaatcaagggccagaacacagcttttagatttagcaacaaggagtttggtgatctcagtgaaagcagcttgatggtgaaatggaggcagaggcagattgcaatgagtgaaacagtgaatggaaagtgaagaaatgatacagataattcttgctaaaagcttggctgttaaaaggaggagagaaacaagactagctgcaaagtgagattgggttgatggagcagttttaaatctcaaaataaagagctttgtgcttttttgattatgaaaataatgtgttaattgtaactaattgaggcaatgaaaaaagataataatatgaaagataaaaatataaaaaccacccagaaataatgatagctaccattttgatacaatatttctacactcctttctatgtatatatacagacacagaaatgcttatatttttattaaaagggattgtactatacctaagctgctttttctagttagtgatatatatggacatctctccatggcaacgagtaattgcagttatattaagttcatgatatttcacaataagggcatatctttgccctttttatttaatcaattcttaattggtgaatgtttgtttccagtttgttgttgttattaacaatgttcccataagcattcctgtacaaaaatgttcacacatttgtctgattttttcttcaggataaaacccaggaggtagaattgctgggttgatagaagagaaaggatgattggcaaattaaagcttcagtagagggtacatgccgagcacaaatgggatcagccctagataccagaaatggcactttctcatttccccttgggacaaaagggagagaggcaataactggtgctgccagagttaaatttgtacgtggagtagcaggaaatcatttgctgaaaatgaaaacagagatgatggtgtagaggtcctgaagagagcaaagaaaatttgaaattgcggctatcagctatggaagagagtgctgaactggaaaacaaaagaagtattgacaattggtatgcttgtaatggcaccgatttgaacgcttgtgccattgttcaccagcagcactcagcagccaagtttggagttttgtagcagaaagacaaataagttagggatttaatatcctggcaaaatggtagacaaaatgaactctgagatccagctgcacagggaaggaagggaagacgggaagaggttagataggaaatacaagagtcaggagactggaagatgttgtgaaatttaagaacacatagagttggagtaaaagtgtaagaaaactagaagggtaagagaccggtcagaaagtaggctatttgaagttaacacttcagaggcagagtagttctgaatggtaacaagaaattgagtgtgcctttgagagtaggttaaaaaacaataggcaactttattgtagctacttctggaacagaagattgtcattaatagttttagaaaactaaaatatatagcatacttatttgtcaattaacaaagaaactatgtatttttaaatgagatttaatgtttattgtagaaaacctggaatcagattactttggcaagcttgaatctaaattatcagtcataagaaatttgaatgaccaagttctcttcattgaccaaggaaatcggcctctatttgaagatatgactgattctgactgtagaggtattttttttaattcgcaaacatagaaatgactagctacttcttcccattctgttttactgcttacattgttccgtgctagtcccaatcctcagatgaaaagtcacaggagtgacaataatttcacttacaggaaactttataaggcatccacgttttttagttggggtaaaaaattggatacaataagacattgctaggggtcatgcctctctgagcctgcctttgaatcaccaatccctttattgtgattgcattaactgtttaaaacctctatagttggatgcttaatccctgcttgttacagctgaaaatgctgatagtttaccaggtgtggtggcatctatctgtaatcctagctacttgggaggctcaagcaggaggattgcttgaggccaggactttgaggctgtagtacactgtgatcgtacctgtgaatagccactgcactccagcctgggtgatatacagaccttgtctctaaaattaaaaaaaaaaaaaaaaaaaaaaaaacttaagaaaagaaaatgatcaagtctactgtgccttccaaaatatgaattccaaatatcaaagttaggctgagttgaagcagtgaatgtgcattctttaaaaatactgaatacttaccttaacatatattttaaatattttatttagcatttaaaagttaaaaacaatcttttagaattcatatctttaaaatactcaaaaaagttgcagcgtgtgtgttgtaatacacattaaactgtggggttgtttgtttgtttgagatgcagtttcactctgtcacccaggctgaagtgcagtgcagtgcagtggtgtgatctcggctcactacaacctccacctcccacgttcaagcgattctcatgcctcagtctcccgagtaggtgggattacaggcatgcaccacttacacccggctaatttttgtatttttagtagagctggggtttcaccatgttggccaggctggtctcaaacccctaacctcaagtgatctgcctgcctcagcctcccaaacaaacaaacaaccccacagtttaatatgtgttacaacacacatgctgcaacttttatgagtataatgatatagattataaaaggttgtttttaacttttaaatgctgggattacaggcatgagccactgtgccaggcctgaactgtgtttttaaaaatgtctgaccagctgtacatagtctcctgcagactggccaagtctcaaagtgggaacaggtgtattaaggactatcctttggttaaatttccgcaaatgttcctgtgcaagaattcttctaactagagttctcatttattatatttatttcagataatgcaccccggaccatatttattataagtatgtataaagatagccagcctagaggtatggctgtaactatctctgtgaagtgtgagaaaatttcaactctctcctgtgagaacaaaattatttcctttaaggtaagactgagccttactttgttttcaatcatgttaatataattaatataattagaaatataacattatttctaatgttaatataagtaatgtaattagaaaactcaaatatcctcagaccaaccttttgtctagaacagaaataacaagaagcagagaaccattaaaatgaatacttactaaaaattatcaaactctttacctattgtgataatgatggtttttctgagcctgtcacagggaagaggagatacaacacttgttttatgacctgcatctcctgaacaatcagtctttatacaaataataatgtagaatacatatgtgagttatacatttaagaataacatgtgactttccagaatgagttctgctatgaagaatgaagctaattatccttctatatttctacacctttgtaaattatgataatattttaatccctagttgttttgttgctgatccttagcctaagtcttagacacaagcttcagcttccagttgatgtatgttatttttaatgttaatctaattgaataaaagttatgagatcagctgtaaaagtaatgctataattatcttcaagccaggtataaagtatttctggcctctactttttctctattattctccattattattctctattatttttctctatttcctccattattgttagataaaccacaattaactatagctacagactgagccagtaagagtagccagggatgcttacaaattggcaatgcttcagaggagaattccatgtcatgaagactctttttgagtggagatttgccaataaataaccgctttcatgcccacccagtccccactgaaagacagttaggatatgaccttagtgaaggtaccaaggggcaacttggtagggagaaaaaagccactctaaaatataatccaagtaagaacagtgcatatgcaacagatacagcccccagacaaatccctcagctatctccctccaaccagagtgccaccccttcaggtgacaatttggagtccccattctagacctgacaggcagcttagttatcaaaatagcataagaggcctgggatggaagggtagggtggaaagggttaagcatgctgttactgaacaacataattagaagggaaggagatggccaagctcaagctatgtgggatagaggaaaactcagctgcagaggcagattcagaaactgggataagtccgaacctacaggtggattcttgttgagggagactggtgaaaatgttaagaagatggaaataatgcttggcacttagtaggaactgggcaaatccatatttgggggagcctgaagtttattcaattttgatggcccttttaaagaaaaagaatgtggctgggcgtggtggctcacacctgtaatcccagcactttgggaggccgaggggggcggatcacctgaagtcaggagttcaagaccagcctgaccaacatggagaaaccccatctctactaaaaatacaaaattagctgggcgtggtggcatatgcctgtaatcccagctactcgggaggctgaggcaggagaatcttttgaacccgggaggcagaggttgcgatgagcctagatcgtgccattgcactccagcctgggcaacaagagcaaaactcggtctcaaaaaaaaaaaaaaaaaaaagtaaaattaccaaaggcataagcttaataatttaattcaaattttaattcaaattttaaaaagggcggggggtggctggaagagatctgtgcaaatgagggaatctgacatttaagcttcatcagcatcatagcaaatctgcttctggaaggaactcaataaatattagttggagggggggagagagtgaggggtggactaggaccagttttagcccttgtctttaatcccttttcctgccactaataaggatcttagcagtggttataaaagtggcctaggttctagataataagatacaacaggccaggcacagtggctcatgcctataatcccagcactttgggagggcaaggcgagtgtctcacttgagatcaggagttcaagaccagcctggccagcatggcgatactctgtctctactaaaaaaaatacaaaaattagccaggcatggtggcatgcacctgtaatcccagctactcgtgagcctgaggcagaagaatcgcttgaaaccaggaggtgtaggctgcagtgagctgagatcgcaccactgcactccagcctgggcgacagaatgagactttgtctcaaaaaaagaaaaagatacaacaggctacccttatgtgctcacctttcactgttgattactagctataaagtcctataaagttctttggtcaagaaccttgacaacactaagagggatttgctttgagaggttactgtcagtgtctgtttcatatatatacatatacatgtatatatgtatctatatccaggcttggccagggttccctcagactttccagtgcacttgggagatgttaggtcaatatcaactttccctggattcagattcaaccccttctgatgtaaaaaaaaaaaaaaaaaaaagaaagaaatccctttccccttggagcactcaagtttcaccaggtagggctttccaagttgggggttctccaaggtcattgggattgctttcacatccatttgctatgtaccttccctatgatggctgggagtggtcaacatcaaaactaggaaagctactgcccaaggatgtccttacctctattctgaaatgtgcaataagtgtgattaaagagattgcctgttctacctatccacactctcgctttcaactgtaactttctttttttctttttttctttttttctttttttttgaaacggagtctcgctctgtcgcccaggctagagtgcagtggcacgatctcagctcactgcaagctctgcctcccgggttcacgccattctcctgcctcaccctcccaagcagctgggactacaggcgcctgccaccatgcccagctaattttttgtatttttagtagagacggggtttcaccgtgttagccaggatggtctcgatctcctgaacttgtgatccgcccgcctcagcctcccaaagtgctgggattacaggcgtgagccatcgcacccggctcaactgtaactttctatactggttcatcttcccctgtaatgttactagagcttttgaagttttggctatggattatttctcatttatacattagatttcagattagttccaaattgatgcccacagcttagggtctcttcctaaattgtatattgtagacagctgcagaagtgggtgccaataggggaactagtttatactttcatcaacttaggacccacacttgttgataaagaacaaaggtcaagagttatgactactgattccacaactgattgagaagttggaggtaaccccgtgacctctgccatccagagtctttcaggctctttgaaggatgaagaaatgctattttaattttggaggtttctctatcagtgcttaggatcatgggaatctgtgctgccatgaggccaaaattaagtccaaaacatctactggttccaggattaacatggaagaaccttaggtggtgcccacatgttctgatccatcctgcaaaatagacatgctgcactaacaggaaaagtgcaggcagcactaccagttggataacctgcaagattatagtttcaagtaatctaaccatttctcacaagaccctattctgtgactgaaacatacaagaatctgcatttggccttctaaggcagggcccagccaaggagaccatattcaggacagaaattcaagactactatggaactggagtgcttggcaggaagacagagtcaaggactgccaactgagccaatacagcaggcttacacaggaacccagggcctagccctacaacaattattgggtctattcactgtaagttttaatttcaggctccactgaaagagtaagctaagattcctggcactttctgtctctctcacagttggctcagaaatgagaactggccaggccaggcatggtggcttacacctggaatcccagcactttgggaggccgaagtgggagggtcacttgaggccaggagttcaggaccagcctaggcaacaaagtgagataccccctgaccccttctctacaaaaataaattttaaaaattagccaaatgtggtggtgtatacttacagtcccagctactcaggaggctgaggcagggggattgcttgagcccaggaattcaaggctgcagtgagctatgatttcaccactgcacttctggctgggcaacagagcgagaccctgtctcaaagcaaaaagaaaaagaaactagaactagcctaagtttgtgggaggaggtcatcatcgtctttagccgtgaatggttattatagaggacagaaattgacattagcccaaaaagcttgtggtctttgctggaactctacttaatcttgagcaaatgtggacaccactcaatgggagaggagagaagtaagctgtttgatgtataggggaaaactagaggcctggaactgaatatgcatcccatgacagggagaataggagattcggagttaagaaggagaggaggtcagtactgctgttcagagattttttttatgtaactcttgagaagcaaaactacttttgttctgtttggtaatatacttcaaaacaaacttcatatattcaaattgttcatgtcctgaaataattaggtaatgtttttttctctataggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagctattaaaatttcatgccgggcgcagtggctcacgcctgtaatcccagccctttgggaggctgaggcgggcagatcaccagaggtcaggtgttcaagaccagcctgaccaacatggtgaaacctcatctctactaaaaatacaaaaaattagctgagtgtagtgacgcatgccctcaatcccagctactcaagaggctgaggcaggagaatcacttgcactccggaggtagaggttgtggtgagccgagattgcaccattgcgctctagcctgggcaacaacagcaaaactccatctcaaaaaataaaataaataaataaacaaataaaaaattcataatgtgaactgtctgaatttttatgtttagaaagattatgagattattagtctataattgtaatggtgaaataaaataaatactagtcttgaaaaacatcattaagaaatgaatgaactttcacaaaagcaaacaaacagactttcccttatttaagtgaataaaataaaataaaataaaataatgtttaaaaaattcatagtttgaaaacattctacattgttaattggcatattaattatacttaatataattatttttaaatcttttgggttattagtcctaatgacaaaagatattgatatttgaactttctaatttttaagaatatcgttaaaccatcaatatttttataaggaggccacttcacttgacaaatttctgaatttcctccaaagtcagtatatttttaaaattcagtttgatcctgaatccagcaatatataaaagggattatatactctggccaactgacattcatcctaggaatgcaaagatggtttaatatcctaaaatcaattaacataacatactatattaataaagtatcaaaacagtattctcatctttttttcttttttcacaattccttggttacactatcatctcaatagatgcagaaaaagcatttgacaaaatccaattcataataaaaattctcaaacttgaaagagaacatcataaaggcatctatgaaaaacctacagctaatatcatacttaacgatgaaaaactgaattattttaccctaagatcaagaataatgcaagcatgtcagctcttgcaacttctattcaacattgtactggaggttctagccagagcaaccatacaataaataaaaataaaaggcacccagattagaaaggaagtctttatttgcagacaacatggttctttatgcagaaaaccgtcaggaatacacacacatgttagaactaataagttcagcaaggttgcaggttgcaatatcaatatgcaaaaatacattgaaggctgggctcagtggagatggcatgtacctttcgtcccagctacttgggaggctgaggtaggaggatcacttgaggtgaggagtttgaggctatagtgcaatgtgatcttgcctgtgaatagccactgcactcgagcctaggcaacaaagtgagaccccgtctccaaaaaaaaaatggtatattggtatttctgtatatgaacaatgaatgatctgaaaacaagaaaattccattcacgatggtattaaaaaaataaaatacaaataaatttagcaaaataattataaaacttgtacatcgaaaatttcaaagcattctgagggaaattaaagatgatctaaataattggagagacattctatgatcactgattggaaaactcattcaatattgttaagataacaattgtccccaaattgatgcatgcattcaatttagtcttcatcaaaattccagcagggtttttgcagaaattgacaagctgtacccaaaatgtatatggaaatgaaaa

SEQ ID NO: 5 - DNA, 22 bp, Artificial Sequence
  PCR Primer
aaggaattgtctcccagtgcat

SEQ ID NO: 6 - DNA, 18 bp, Artificial Sequence
  PCR Primer
caggtggcagccgcttta

SEQ ID NO: 7 - DNA, 22 bp, Artificial Sequence
  PCR Probe
ttgccctcctggctgccaactc

SEQ ID NO: 8 - DNA, 19 bp, Artificial Sequence
  PCR Primer
gaaggtgaaggtcggagtc

SEQ ID NO: 9 - DNA, 20 bp, Artificial Sequence
  PCR Primer
gaagatggtgatgggatttc

SEQ ID NO: 10 - DNA, 20 bp, Artificial Sequence
  PCR Probe
caagcttcccgttctcagcc

SEQ ID NO: 11 - DNA, 1102 bp, H. sapiens
  CDS (178)...(759)
gcctggacagtcagcaaggaattgtctcccagtgcattttgccctcctggctgccaactctggctgctaaagcggctgccacctgctgcagtctacacagcttcgggaagaggaaaggaacctcagaccttccagatcgcttcctctcgcaacaaactatttgtcgcaggaataaagatggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagctattaaaatttcatgccgggcgcagtggctcacgcctgtaatcccagccctttgggaggctgaggcgggcagatcaccagaggtcaggtgttcaagaccagcctgaccaacatggtgaaacctcatctctactaaaaatactaaaaattagctgagtgtagtgacgcatgccctcaatcccagctactcaagaggctgaggcaggagaatcacttgcactccggaggtagaggttgtggtgagccgagattgcaccattgcgctctagcctgggcaacaacagcaaaactccatctcaaaaaataaaataaataaataaacaaataaaaaattcataatgtg

SEQ ID NO: 12 - skipped

SEQ ID NO: 13 - DNA, 20 bp, Artificial Sequence
  Antisense Oligonucleotide
tatctctacagtcagaatca

SEQ ID NO: 14 - DNA, 20 bp, Artificial Sequence
  Antisense Oligonucleotide
caattgtcttctactggttc

SEQ ID NO: 15 - DNA, 20 bp, Artificial Sequence
  Antisense Oligonucleotide
aatttagattcaagcttgcc

SEQ ID NO: 16 - DNA, 20 bp, Artificial Sequence
  Antisense Oligonucleotide
atgtcctgggacacttctct

SEQ ID NO: 17 - DNA, 20 bp, Artificial Sequence
  Antisense Oligonucleotide
gccgctttagcagccagagt

SEQ ID NO: 18 - DNA, 20 bp, Artificial Sequence
  Antisense Oligonucleotide
cagcaggtggcagccgcttt

SEQ ID NO: 19 - DNA, 20 bp, Artificial Sequence
  Antisense Oligonucleotide
tctgattccaggttttcatc

SEQ ID NO: 20 - DNA, 20 bp, Artificial Sequence
  Antisense Oligonucleotide
gctatctttatacatactta

SEQ ID NO: 21 - DNA, 20 bp, Artificial Sequence
  Antisense Oligonucleotide
tccttgctgactgtccaggc

SEQ ID NO: 22 - DNA, 20 bp, Artificial Sequence
  Antisense Oligonucleotide
tttcacaagctagaaagtat

SEQ ID NO: 23 - DNA, 20 bp, Artificial Sequence
  Antisense Oligonucleotide
atgcaattgtcttctactgg

SEQ ID NO: 24 - DNA, 20 bp, Artificial Sequence
  Antisense Oligonucleotide
catcatcttcagctataaag

SEQ ID NO: 25 - DNA, 20 bp, Artificial Sequence
  Antisense Oligonucleotide
cctcttcccgaagctgtgta

SEQ ID NO: 26 - DNA, 20 bp, Artificial Sequence
  Antisense Oligonucleotide
aggaaataattttgttctca

SEQ ID NO: 27 - DNA, 20 bp, Artificial Sequence
  Antisense Oligonucleotide
ctctaggctggctatcttta

SEQ ID NO: 28 - DNA, 20 bp, Artificial Sequence
  Antisense Oligonucleotide
tgaaattttctcacacttca

SEQ ID NO: 29 - DNA, 20 bp, Artificial Sequence
  Antisense Oligonucleotide
gccatctttattcctgcgac

SEQ ID NO: 30 - DNA, 20 bp, Artificial Sequence
  Antisense Oligonucleotide
gagacaattccttgctgact

SEQ ID NO: 31 - DNA, 20 bp, Artificial Sequence
  Antisense Oligonucleotide
aatctgattccaggttttca

SEQ ID NO: 32 - DNA, 20 bp, Artificial Sequence
  Antisense Oligonucleotide
catcttattatcatgtcctg

SEQ ID NO: 33 - DNA, 20 bp, Artificial Sequence
  Antisense Oligonucleotide
aggtctgaggttcctttcct

SEQ ID NO: 34 - DNA, 20 bp, Artificial Sequence
  Antisense Oligonucleotide
cttctactggttcagcagcc

SEQ ID NO: 35 - DNA, 20 bp, Artificial Sequence
  Antisense Oligonucleotide
tagttacagccatacctcta

SEQ ID NO: 36 - DNA, 20 bp, Artificial Sequence
  Antisense Oligonucleotide
ttcatcatcttcagctataa

SEQ ID NO: 37 - DNA, 20 bp, Artificial Sequence
  Antisense Oligonucleotide
agtatccttcgtatgatgaa

SEQ ID NO: 38 - DNA, 20 bp, Artificial Sequence
  Antisense Oligonucleotide
ccatctttattcctgcgaca

SEQ ID NO: 39 - DNA, 20 bp, Artificial Sequence
  Antisense Oligonucleotide
tgttgcgagaggaagcgatc

SEQ ID NO: 40 - DNA, 20 bp, Artificial Sequence
  Antisense Oligonucleotide
agactgcagcaggtggcagc

SEQ ID NO: 41 - DNA, 20 bp, Artificial Sequence
  Antisense Oligonucleotide
agctagaaagtatccttcgt

SEQ ID NO: 42 - DNA, 20 bp, Artificial Sequence
  Antisense Oligonucleotide
aattgcatcttattatcatg

SEQ ID NO: 43 - DNA, 20 bp, Artificial Sequence
  Antisense Oligonucleotide
gtcatatcttcaaatagagg

SEQ ID NO: 44 - DNA, 20 bp, Artificial Sequence
  Antisense Oligonucleotide
aggagggcaaaatgcactgg

SEQ ID NO: 45 - DNA, 20 bp, Artificial Sequence
  Antisense Oligonucleotide
tttccttggtcaatgaagag

SEQ ID NO: 46 - DNA, 20 bp, Artificial Sequence
  Antisense Oligonucleotide
cgttttgaacagtgaacatt

SEQ ID NO: 47 - DNA, 20 bp, Artificial Sequence
  Antisense Oligonucleotide
tgcgacaaatagtttgttgc

SEQ ID NO: 48 - DNA, 20 bp, Artificial Sequence
  Antisense Oligonucleotide
tcttattatcatgtcctggg

SEQ ID NO: 49 - DNA, 20 bp, Artificial Sequence
  Antisense Oligonucleotide
caaatagtttgttgcgagag

SEQ ID NO: 50 - DNA, 20 bp, Artificial Sequence
  Antisense Oligonucleotide
cacaaagttgatgcaattgt

SEQ ID NO: 51 - DNA, 20 bp, Artificial Sequence
  Antisense Oligonucleotide
cattgccacaaagttgatgc

SEQ ID NO: 52 - DNA, 20 bp, Artificial Sequence
  Antisense Oligonucleotide
caataaatttcattgccaca

SEQ ID NO: 53 - DNA, 20 bp, Artificial Sequence
  Antisense Oligonucleotide
tataaagtaaagcgtattgt

SEQ ID NO: 54 - DNA, 20 bp, Artificial Sequence
  Antisense Oligonucleotide
caagcttgccaaagtaatct

SEQ ID NO: 55 - DNA, 20 bp, Artificial Sequence
  Antisense Oligonucleotide
tatgactgataatttagatt

SEQ ID NO: 56 - DNA, 20 bp, Artificial Sequence
  Antisense Oligonucleotide
aatttcttatgactgataat

SEQ ID NO: 57 - DNA, 20 bp, Artificial Sequence
  Antisense Oligonucleotide
attcaaatttcttatgactg

SEQ ID NO: 58 - DNA, 20 bp, Artificial Sequence
  Antisense Oligonucleotide
acttggtcattcaaatttct

SEQ ID NO: 59 - DNA, 20 bp, Artificial Sequence
  Antisense Oligonucleotide
atgaagagaacttggtcatt

SEQ ID NO: 60 - DNA, 20 bp, Artificial Sequence
  Antisense Oligonucleotide
gtcagaatcagtcatatctt

SEQ ID NO: 61 - DNA, 20 bp, Artificial Sequence
  Antisense Oligonucleotide
ttataataaatatggtccgg

SEQ ID NO: 62 - DNA, 20 bp, Artificial Sequence
  Antisense Oligonucleotide
cacttcacagagatagttac

SEQ ID NO: 63 - DNA, 20 bp, Artificial Sequence
  Antisense Oligonucleotide
ctcacaggagagagttgaaa

SEQ ID NO: 64 - DNA, 20 bp, Artificial Sequence
  Antisense Oligonucleotide
gattcatttccttaaaggaa

SEQ ID NO: 65 - DNA, 20 bp, Artificial Sequence
  Antisense Oligonucleotide
gttatcaggaggattcattt

SEQ ID NO: 66 - DNA, 20 bp, Artificial Sequence
  Antisense Oligonucleotide
gtatccttgatgttatcagg

SEQ ID NO: 67 - DNA, 20 bp, Artificial Sequence
  Antisense Oligonucleotide
aagaatatgatgtcactttt

SEQ ID NO: 68 - DNA, 20 bp, Artificial Sequence
  Antisense Oligonucleotide
tgaaagaatatgatgtcact

SEQ ID NO: 69 - DNA, 20 bp, Artificial Sequence
  Antisense Oligonucleotide
cttctctgaaagaatatgat

SEQ ID NO: 70 - DNA, 20 bp, Artificial Sequence
  Antisense Oligonucleotide
cacttctctgaaagaatatg

SEQ ID NO: 71 - DNA, 20 bp, Artificial Sequence
  Antisense Oligonucleotide
tgatgaagattcaaattgca

SEQ ID NO: 72 - DNA, 20 bp, Artificial Sequence
  Antisense Oligonucleotide
gtatgatgaagattcaaatt

SEQ ID NO: 73 - DNA, 20 bp, Artificial Sequence
  Antisense Oligonucleotide
agctagtcttcgttttgaac

SEQ ID NO: 74 - DNA, 20 bp, Artificial Sequence
  Antisense Oligonucleotide
attttaatagctagtcttcg

SEQ ID NO: 75 - DNA, 20 bp, Artificial Sequence
  Antisense Oligonucleotide
tgtccaggcagtcgccaggg

SEQ ID NO: 76 - DNA, 20 bp, Artificial Sequence
  Antisense Oligonucleotide
gctgactgtccaggcagtcg

SEQ ID NO: 77 - DNA, 20 bp, Artificial Sequence
  Antisense Oligonucleotide
tgttcattgaatttggcaat

SEQ ID NO: 78 - DNA, 20 bp, Artificial Sequence
  Antisense Oligonucleotide
aatacatcccttagcacttg

SEQ ID NO: 79 - DNA, 20 bp, Artificial Sequence
  Antisense Oligonucleotide
atctttattcctaatgaaag

SEQ ID NO: 80 - DNA, 20 bp, Artificial Sequence
  Antisense Oligonucleotide
ttagccttacctataaagta

SEQ ID NO: 81 - DNA, 20 bp, Artificial Sequence
  Antisense Oligonucleotide
tcagtcttaccttaaaggaa

SEQ ID NO: 82 - DNA, 20 bp, Artificial Sequence
  Antisense Oligonucleotide
catcaactggaagctgaagc

SEQ ID NO: 83 - DNA, 20 bp, Artificial Sequence
  Antisense Oligonucleotide
ctaagttgatgaaagtataa

SEQ ID NO: 84 - DNA, 20 bp, Artificial Sequence
  Antisense Oligonucleotide
gattcatttcctatagagaa

SEQ ID NO: 85 - DNA, 20 bp, H. sapiens
gaaccagtagaagacaattg

SEQ ID NO: 86 - DNA, 20 bp, H. sapiens
agagaagtgtcccaggacat

SEQ ID NO: 87 - DNA, 20 bp, H. sapiens
taagtatgtataaagatagc

SEQ ID NO: 88 - DNA, 20 bp, H. sapiens
atactttctagcttgtgaaa

SEQ ID NO: 89 - DNA, 20 bp, H. sapiens
ccagtagaagacaattgcat

SEQ ID NO: 90 - DNA, 20 bp, H. sapiens
tacacagcttcgggaagagg

SEQ ID NO: 91 - DNA, 20 bp, H. sapiens
taaagatagccagcctagag

SEQ ID NO: 92 - DNA, 20 bp, H. sapiens
tgaagtgtgagaaaatttca

SEQ ID NO: 93 - DNA, 20 bp, H. sapiens
agtcagcaaggaattgtctc

SEQ ID NO: 94 - DNA, 20 bp, H. sapiens
ggctgctgaaccagtagaag

SEQ ID NO: 95 - DNA, 20 bp, H. sapiens
ttcatcatacgaaggatact

SEQ ID NO: 96 - DNA, 20 bp, H. sapiens
gctgccacctgctgcagtct

SEQ ID NO: 97 - DNA, 20 bp, H. sapiens
acgaaggatactttctagct

SEQ ID NO: 98 - DNA, 20 bp, H. sapiens
aatgttcactgttcaaaacg

SEQ ID NO: 99 - DNA, 20 bp, H. sapiens
gcaacaaactatttgtcgca

SEQ ID NO: 100 - DNA, 20 bp, H. sapiens
cccaggacatgataataaga

SEQ ID NO: 101 - DNA, 20 bp, H. sapiens
acaattgcatcaactttgtg

SEQ ID NO: 102 - DNA, 20 bp, H. sapiens
gcatcaactttgtggcaatg

SEQ ID NO: 103 - DNA, 20 bp, H. sapiens
tgtggcaatgaaatttattg

SEQ ID NO: 104 - DNA, 20 bp, H. sapiens
acaatacgctttactttata

SEQ ID NO: 105 - DNA, 20 bp, H. sapiens
agattactttggcaagcttg

SEQ ID NO: 106 - DNA, 20 bp, H. sapiens
aatctaaattatcagtcata

SEQ ID NO: 107 - DNA, 20 bp, H. sapiens
cagtcataagaaatttgaat

SEQ ID NO: 108 - DNA, 20 bp, H. sapiens
agaaatttgaatgaccaagt

SEQ ID NO: 109 - DNA, 20 bp, H. sapiens
aatgaccaagttctcttcat

SEQ ID NO: 110 - DNA, 20 bp, H. sapiens
aagatatgactgattctgac

SEQ ID NO: 111 - DNA, 20 bp, H. sapiens
ccggaccatatttattataa

SEQ ID NO: 112 - DNA, 20 bp, H. sapiens
gtaactatctctgtgaagtg

SEQ ID NO: 113 - DNA, 20 bp, H. sapiens
tttcaactctctcctgtgag

SEQ ID NO: 114 - DNA, 20 bp, H. sapiens
ttcctttaaggaaatgaatc

SEQ ID NO: 115 - DNA, 20 bp, H. sapiens
aaatgaatcctcctgataac

SEQ ID NO: 116 - DNA, 20 bp, H. sapiens
cctgataacatcaaggatac

SEQ ID NO: 117 - DNA, 20 bp, H. sapiens
aaaagtgacatcatattctt

SEQ ID NO: 118 - DNA, 20 bp, H. sapiens
agtgacatcatattctttca

SEQ ID NO: 119 - DNA, 20 bp, H. sapiens
atcatattctttcagagaag

SEQ ID NO: 120 - DNA, 20 bp, H. sapiens
catattctttcagagaagtg

SEQ ID NO: 121 - DNA, 20 bp, H. sapiens
tgcaatttgaatcttcatca

SEQ ID NO: 122 - DNA, 20 bp, H. sapiens
aatttgaatcttcatcatac

SEQ ID NO: 123 - DNA, 20 bp, H. sapiens
gttcaaaacgaagactagct

SEQ ID NO: 124 - DNA, 20 bp, H. sapiens
cgaagactagctattaaaat

SEQ ID NO: 125 - DNA, 20 bp, H. sapiens
ccctggcgactgcctggaca

SEQ ID NO: 126 - DNA, 20 bp, H. sapiens
cgactgcctggacagtcagc

SEQ ID NO: 127 - DNA, 20 bp, H. sapiens
attgccaaattcaatgaaca

SEQ ID NO: 128 - DNA, 20 bp, H. sapiens
caagtgctaagggatgtatt

SEQ ID NO: 129 - DNA, 20 bp, H. sapiens
ttcctttaaggtaagactga

SEQ ID NO: 130 - DNA, 20 bp, H. sapiens
gcttcagcttccagttgatg

(19) United States
(12) Patent Application Publication
(10) Pub. No.: US 2004/0115636 A1
(43) Pub. Date: Jun. 17, 2004

(54) Modulation of interleukin 18 expression

(75) Inventors: Brenda F. Baker, Carlsbad, CA (US); Kenneth W. Dobie, Del Mar, CA (US)

Correspondence Address: MARSHALL, GERSTEIN & BORUN, 6300 SEARS TOWER, 233 SOUTH WACKER DRIVE, CHICAGO, IL, 60606-6357, US

(73) Assignee: Isis Pharmaceuticals Inc.

(21) Appl. No.: 10/317,478

(22) Filed: Dec. 11, 2002

(51) Int. Cl. C12Q 1/68; C07H 21/04; A61K 48/00

(52) U.S. Cl. 435/6; 514/44; 536/23.5

(57) ABSTRACT

Compounds, compositions and methods are provided for modulating the expression of Interleukin 18. The compositions comprise oligonucleotides, targeted to nucleic acid encoding Interleukin 18. Methods of using these compounds for modulation of Interleukin 18 expression and for diagnosis and treatment of disease associated with expression of Interleukin 18 are provided.

### FIELD OF THE INVENTION

The present invention provides compositions and methods for modulating the expression of Interleukin 18. In particular, this invention relates to compounds, particularly oligonucleotide compounds, which, in preferred embodiments, hybridize with nucleic acid molecules encoding Interleukin 18. Such compounds are shown herein to modulate the expression of Interleukin 18.

### BACKGROUND OF THE INVENTION

Inflammation is a physiological response to various stimuli such as infection or trauma that results in release of cytokines by macrophages and lymphocytes. The inflammatory response is characterized by increased capillary blood flow and permeability allowing various cells and fluid to leave the capillaries and enter the affected region resulting in swelling, redness, heat and pain. The increased capillary blood flow and permeability enables cellular and humoral components of the immune system such as cytokines and other mediators to enter the affected area and assist in the removal of bacteria and repair of connective tissues (Kulmatycki and Jamali, Cytokine, 2001, 14, 1-10).

There are many classes of cytokines such as chemokines, interleukins (IL), tumor necrosis factors (TNF), interferons (IFN), hematoproteins, colony stimulating factors as well as neurotrophins and growth factors. The interleukins have both inflammatory and anti-inflammatory activities (Kulmatycki and Jamali, Cytokine, 2001, 14, 1-10).

Interleukin 18 (also known as IL-18, interferon-gamma inducing factor, IGIF and IFN-gamma inducer) was originally identified as an interferon-gamma-inducing factor in splenocytes, hepatic lymphocytes and type 1 helper T cell clones (Sugawara, Microbes Infect., 2000, 2, 1257-1263). Interleukin 18 has been cloned (Okamura et al., Nature, 1995, 378, 88-91) and mapped to chromosome 11q22.2-q22.3 (Nolan et al., Genomics, 1998, 51, 161-163). Interleukin 18 mRNA is most abundant in pancreas, kidney and skeletal muscle with detectable levels also found in liver and lung tissues (Ushio et al., J. Immunol., 1996, 156, 4274-4279).

Nucleic acid sequences encoding interleukin 18 are disclosed and claimed in U.S. Pat. No. 6,060,283 and Japanese Patents JP10080288 and JP10007699 (Okura et al., 2000; Shinpei et al., 1998; Takanori et al., 1998)

The gene is composed of six exons (one of which is not translated) and five introns spanning approximately 19.5 kb. The nucleotide sequences at the exon/intron junctions are in accordance with the eukaryotic splice donor-acceptor site sequence AG/GU (Kalina et al., Scand. J. Immunol., 2000, 52, 525-530).

The main coding sequence of interleukin 18 is composed of exons 1-5 (Kalina et al., Scand. J. Immunol., 2000, 52, 525-530). However, two possible mRNA variants have been isolated, one which uses exons 1-4, and a short exon 5 (exon 5a) herein designated IL-18 variant I, and one which starts within exon 2 (exon 2a), uses exons 3-4, and a short exon 5 (exon 5b) herein designated IL-18 variant II.

Interleukin 18 augments cytotoxicity of natural killer (NK) cells and proliferation of T cells. It stimulates Th₁ cells to produce interleukin 2 and interferon-gamma, an effect which is augmented in a synergistic manner by interleukin 12 (Golab, Cytokine, 2000, 12, 332-338).

Interleukin 18 knockout mice have an impaired NK cell activity, a reduced production of interferon-gamma and a diminished Th₁ response (Golab, Cytokine, 2000, 12, 332-338).

Increased levels of interleukin 18 are observed in the serum of patients with schizophrenia, pulmonary sarcoidosis, and Graves disease (Miyauchi et al., Thyroid, 2000, 10, 815-819; Shigehara et al., Am. J. Respir. Crit. Care Med., 2000, 162, 1979-1982; Tanaka et al., Psychiatry Res., 2000, 96, 75-80) as well as the mucosal intestinal cells and lamina propria mononuclear cells from patients with Crohn's disease (Monteleone et al., J. Immunol., 1999, 163, 143-147).

Interleukin 18 has been found to increase expression of vascular cell adhesion molecule-1 (VCAM-1) which leads to the adherence of melanoma cells, a critical step in the initiation of metastasis (Vidal-Vanaclocha et al., Proc. Natl. Acad. Sci. U.S.A., 2000, 97, 734-739).

The inhibition of interleukin 18 may prove to be a useful strategy for therapeutic intervention in a wide variety of pathological conditions. Neutralization of interleukin 18 activity has been reported for six different isoforms of interleukin 18 binding protein (Dinarello, Ann. Rheum. Dis., 2000, 59 Suppl 1, i17-20). Dinarello has indicated that interleukin 18 binding protein may find therapeutic use for treatment of rheumatoid arthritis, Crohn's disease, toxic liver damage, lupus erythematosus, Still's disease, psoriasis, graft vs host, transplant rejection, multiple sclerosis and septic shock (Dinarello, Ann. Rheum. Dis., 2000, 59 Suppl 1, i17-20).

Examples of using antibodies to inhibit interleukin 18 production and/or action are well represented in the art. For example, antibodies to interleukin 18 are disclosed and claimed in PCT publications WO 01/07480 and WO 00/56771 (Dinarello, 2001; Ho et al., 2000). Additionally disclosed and claimed in PCT publication WO 01/07489 are antagonists of interleukin 18 which compete with interleukin 18 and block the interleukin 18 receptor, and interleukin 18 binding proteins (Dinarello, 2001).

Disclosed and claimed in European patent EP974600 are artificially produced peptides capable of neutralizing the biological activities of interleukin 18, comprising a part or the whole of a variable region in an anti-interleukin 18 antibody (Nishida et al., 1999).

A functional effect of interleukin 18 on B16F10 melanoma cells has been shown by reduction of Fas ligand expression in cells treated with anti-interleukin 18 antibody or transfected with interleukin 18 antisense RNA (Cho et al., Cancer Res., 2000, 60, 2703-2709).

An interleukin 18 antisense oligonucleotide has been used to decrease expression of interleukin 18 in an investigation of effects of interleukin 18 on proliferation in the J6-1 human leukemia cell line (Wang et al., Zhonghua Yixue Zazhi (Beijing, China), 2001, 81, 597-600).

An antisense phosphorothioate oligonucleotide targeting the initiation site of human interleukin 18 was used to down-regulate interleukin 18 expression in mucosal intestinal cells and lamina propria mononuclear cells from patients with Crohn's disease (Monteleone et al., J. Immunol., 1999, 163, 143-147).

Currently, there are no known therapeutic agents that effectively inhibit the synthesis of interleukin 18. To date, investigative strategies aimed at modulating interleukin 18 expression have involved the use of antibodies and gene knock-outs in mice. Consequently, there remains a long felt need for additional agents capable of effectively inhibiting interleukin 18 function.

Antisense technology is emerging as an effective means for reducing the expression of specific gene products and may therefore prove to be uniquely useful in a number of therapeutic, diagnostic, and research applications for the modulation of expression of interleukin 18.

The present invention provides compositions and methods for modulating expression of interleukin 18.

### SUMMARY OF THE INVENTION

The present invention is directed to compounds, especially nucleic acid and nucleic acid-like oligomers, which are targeted to a nucleic acid encoding Interleukin 18, and which modulate the expression of Interleukin 18. Pharmaceutical and other compositions comprising the compounds of the invention are also provided. Further provided are methods of screening for modulators of Interleukin 18 and methods of modulating the expression of Interleukin 18 in cells, tissues or animals comprising contacting said cells, tissues or animals with one or more of the compounds or compositions of the invention. Methods of treating an animal, particularly a human, suspected of having or being prone to a disease or condition associated with expression of Interleukin 18 are also set forth herein. Such methods comprise administering a therapeutically or prophylactically effective amount of one or more of the compounds or compositions of the invention to the person in need of treatment.

### DETAILED DESCRIPTION OF THE INVENTION

A. Overview of the Invention

The present invention employs compounds, preferably oligonucleotides and similar species for use in modulating the function or effect of nucleic acid molecules encoding Interleukin 18. This is accomplished by providing oligonucleotides which specifically hybridize with one or more nucleic acid molecules encoding Interleukin 18. As used herein, the terms “target nucleic acid” and “nucleic acid molecule encoding Interleukin 18” have been used for convenience to encompass DNA encoding Interleukin 18, RNA (including pre-mRNA and mRNA or portions thereof) transcribed from such DNA, and also cDNA derived from such RNA. The hybridization of a compound of this invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of Interleukin 18. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

In the present invention the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated.

“Complementary,” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is preferred that the antisense compounds of the present invention comprise at least 70% sequence complementarity to a target region within the target nucleic acid, more preferably that they comprise 90% sequence complementarity and even more preferably comprise 95% sequence complementarity to the target region within the target nucleic acid sequence to which they are targeted. For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

B. Compounds of the Invention

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo and Kempheus, Cell, 1995, 81, 611-620). Montgomery et al. have shown that the primary interference effects of dsRNA are posttranscriptional (Montgomery et al., Proc. Natl. Acad. Sci. USA, 1998, 95, 15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391, 806-811). Recently, it has been shown that it is, in fact, the single-stranded RNA oligomers of antisense polarity of the dsRNAs which are the potent inducers of RNAi (Tijsterman et al., Science, 2002, 295, 694-697).

In the context of this invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention comprehends other families of compounds as well, including but not limited to oligonucleotide analogs and mimetics such as those described herein.

The compounds in accordance with this invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In one preferred embodiment, the compounds of the invention are 12 to 50 nucleobases in length. One having ordinary skill in the art will appreciate that this embodies compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another preferred embodiment, the compounds of the invention are 15 to 30 nucleobases in length. One having ordinary skill in the art will appreciate that this embodies compounds of 15, 16, 17, 18, 19, 20, 21, 22, 23, 24, 25, 26, 27, 28, 29, or 30 nucleobases in length.

Particularly preferred compounds are oligonucleotides from about 12 to about 50 nucleobases, even more preferably those comprising from about 15 to about 30 nucleobases.

Antisense compounds 8-80 nucleobases in length comprising a stretch of at least eight (8) consecutive nucleobases selected from within the illustrative antisense compounds are considered to be suitable antisense compounds as well.

Exemplary preferred antisense compounds include oligonucleotide sequences that comprise at least the 8 consecutive nucleobases from the 5′-terminus of one of the illustrative preferred antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Similarly preferred antisense compounds are represented by oligonucleotide sequences that comprise at least the 8 consecutive nucleobases from the 3′-terminus of one of the illustrative preferred antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). One having skill in the art armed with the preferred antisense compounds illustrated herein will be able, without undue experimentation, to identify further preferred antisense compounds.

C. Targets of the Invention

“Targeting” an antisense compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent. In the present invention, the target nucleic acid encodes Interleukin 18.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding Interleukin 18, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense compounds of the present invention.

The open reading frame (ORF) or “coding region,” which is known in the art to refer to the region between the translation initiation codon and the translation termination codon, is also a region which may be targeted effectively. Within the context of the present invention, a preferred region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also preferred target nucleic acids.

The locations on the target nucleic acid to which the preferred antisense compounds hybridize are hereinbelow referred to as “preferred target segments.” As used herein the term “preferred target segment” is defined as at least an 8-nucleobase portion of a target region to which an active antisense compound is targeted. While not wishing to be bound by theory, it is presently believed that these target segments represent portions of the target nucleic acid which are accessible for hybridization.

While the specific sequences of certain preferred target segments are set forth herein, one of skill in the art will recognize that these serve to illustrate and describe particular embodiments within the scope of the present invention. Additional preferred target segments may be identified by one having ordinary skill.

Target segments 8-80 nucleobases in length comprising a stretch of at least eight (8) consecutive nucleobases selected from within the illustrative preferred target segments are considered to be suitable for targeting as well.

Target segments can include DNA or RNA sequences that comprise at least the 8 consecutive nucleobases from the 5′-terminus of one of the illustrative preferred target segments (the remaining nucleobases being a consecutive stretch of the same DNA or RNA beginning immediately upstream of the 5′-terminus of the target segment and continuing until the DNA or RNA contains about 8 to about 80 nucleobases). Similarly preferred target segments are represented by DNA or RNA sequences that comprise at least the 8 consecutive nucleobases from the 3′-terminus of one of the illustrative preferred target segments (the remaining nucleobases being a consecutive stretch of the same DNA or RNA beginning immediately downstream of the 3′-terminus of the target segment and continuing until the DNA or RNA contains about 8 to about 80 nucleobases). One having skill in the art armed with the preferred target segments illustrated herein will be able, without undue experimentation, to identify further preferred target segments.

Once one or more target regions, segments or sites have been identified, antisense compounds are chosen which are sufficiently complementary to the target, i.e., hybridize sufficiently well and with sufficient specificity, to give the desired effect.

D. Screening and Target Validation

In a further embodiment, the “preferred target segments” identified herein may be employed in a screen for additional compounds that modulate the expression of Interleukin 18. “Modulators” are those compounds that decrease or increase the expression of a nucleic acid molecule encoding Interleukin 18 and which comprise at least an 8-nucleobase portion which is complementary to a preferred target segment. The screening method comprises the steps of contacting a preferred target segment of a nucleic acid molecule encoding Interleukin 18 with one or more candidate modulators, and selecting for one or more candidate modulators which decrease or increase the expression of a nucleic acid molecule encoding Interleukin 18. Once it is shown that the candidate modulator or modulators are capable of modulating (e.g. either decreasing or increasing) the expression of a nucleic acid molecule encoding Interleukin 18, the modulator may then be employed in further investigative studies of the function of Interleukin 18, or for use as a research, diagnostic, or therapeutic agent in accordance with the present invention.

The preferred target segments of the present invention may be also be combined with their respective complementary antisense compounds of the present invention to form stabilized double-stranded (duplexed) oligonucleotides.

Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The compounds of the present invention can also be applied in the areas of drug discovery and target validation. The present invention comprehends the use of the compounds and preferred target segments identified herein in drug discovery efforts to elucidate relationships that exist between Interleukin 18 and a disease state, phenotype, or condition. These methods include detecting or modulating Interleukin 18 comprising contacting a sample, tissue, cell, or organism with the compounds of the present invention, measuring the nucleic acid or protein level of Interleukin 18 and/or a related phenotypic or chemical endpoint at some time after treatment, and optionally comparing the measured value to a non-treated sample or sample treated with a further compound of the invention. These methods can also be performed in parallel or in combination with other experiments to determine the function of unknown genes for the process of target validation or to determine the validity of a particular gene product as a target for treatment or prevention of a particular disease, condition, or phenotype.

E. Kits, Research Reagents, Diagnostics, and Therapeutics

The compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compounds of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The compounds of the invention are useful for research and diagnostics, because these compounds hybridize to nucleic acids encoding Interleukin 18. For example, oligonucleotides that are shown to hybridize with such efficiency and under such conditions as disclosed herein as to be effective Interleukin 18 inhibitors will also be effective primers or probes under conditions favoring gene amplification or detection, respectively. These primers and probes are useful in methods requiring the specific detection of nucleic acid molecules encoding Interleukin 18 and in the amplification of said nucleic acid molecules for detection or for use in further studies of Interleukin 18. Hybridization of the antisense oligonucleotides, particularly the primers and probes, of the invention with a nucleic acid encoding Interleukin 18 can be detected by means known in the art. Such means may include conjugation of an enzyme to the oligonucleotide, radiolabelling of the oligonucleotide or any other suitable detection means. Kits using such detection means for detecting the level of Interleukin 18 in a sample may also be prepared.

The specificity and sensitivity of antisense is also harnessed by those of skill in the art for therapeutic uses. Antisense compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense compounds can be useful therapeutic modalities that can be configured to be useful in treatment regimes for the treatment of cells, tissues and animals, especially humans.

For therapeutics, an animal, preferably a human, suspected of having a disease or disorder which can be treated by modulating the expression of Interleukin 18 is treated by administering antisense compounds in accordance with this invention. For example, in one non-limiting embodiment, the methods comprise the step of administering to the animal in need of treatment, a therapeutically effective amount of a Interleukin 18 inhibitor. The Interleukin 18 inhibitors of the present invention effectively inhibit the activity of the Interleukin 18 protein or inhibit the expression of the Interleukin 18 protein. In one embodiment, the activity or expression of Interleukin 18 in an animal is inhibited by about 10%. Preferably, the activity or expression of Interleukin 18 in an animal is inhibited by about 30%. More preferably, the activity or expression of Interleukin 18 in an animal is inhibited by 50% or more.

For example, the reduction of the expression of Interleukin 18 may be measured in serum, adipose tissue, liver or any other body fluid, tissue or organ of the animal. Preferably, the cells contained within said fluids, tissues or organs being analyzed contain a nucleic acid molecule encoding Interleukin 18 protein and/or the Interleukin 18 protein itself.

The compounds of the invention can be utilized in pharmaceutical compositions by adding an effective amount of a compound to a suitable pharmaceutically acceptable diluent or carrier. Use of the compounds and methods of the invention may also be useful prophylactically.

F. Modifications

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Modified Internucleoside Linkages (Backbones)

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Modified Sugar and Internucleoside Linkages-Mimetics

In other preferred oligonucleotide mimetics, both the sugar and the internucleoside linkage (i.e. the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science, 1991, 254, 1497-1500.

Preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified Sugars

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples hereinbelow.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

A further preferred modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is preferably a methylene (—CH₂—)ₙ group bridging the 2′ oxygen atom and the 4′ carbon atom wherein n is 1 or 2. LNAs and preparation thereof are described in WO 98/39352 and WO 99/14226.

Natural and Modified Nucleobases

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′, 2′: 4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos.: 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Conjugates

Another modification of the oligonucleotides of the invention involves chemically linking to the oligonucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application PCT/US92/09196, filed Oct. 23, 1992, and U.S. Pat. No. 6,287,860, the entire disclosure of which are incorporated herein by reference. Conjugate moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

Chimeric Compounds

It is not necessary for all positions in a given compound to be uniformly modified, and in fact more than one of the aforementioned modifications may be incorporated in a single compound or even at a single nucleoside within an oligonucleotide.

The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide-mediated inhibition of gene expression. The cleavage of RNA:RNA hybrids can, in like fashion, be accomplished through the actions of endoribonucleases, such as RNAseL which cleaves both cellular and viral RNA. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

G. Formulations

The compounds of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions of the present invention may also include surfactants. The use of surfactants in drug products, formulations and in emulsions is well known in the art. Surfactants and their uses are further described in U.S. Pat. No. 6,287,860, which is incorporated herein in its entirety.

In one embodiment, the present invention employs various penetration enhancers to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally, in granular form including sprayed dried particles, or complexed to form micro or nanoparticles. Oligonucleotide complexing agents and their uses are further described in U.S. Pat. No. 6,287,860, which is incorporated herein in its entirety. Oral formulations for oligonucleotides and their preparation are described in detail in U.S. application Ser. Nos. 09/108,673 (filed Jul. 1, 1998), 09/315,298 (filed May 20, 1999) and 10/071,822, filed Feb. 8, 2002, each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more oligomeric compounds and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of antisense compounds and other non-antisense drugs are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Alternatively, compositions of the invention may contain two or more antisense compounds targeted to different regions of the same nucleic acid target. Numerous examples of antisense compounds are known in the art. Two or more combined compounds may be used together or sequentially.

H. Dosing

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

While the present invention has been described with specificity in accordance with certain of its preferred embodiments, the following examples serve only to illustrate the invention and are not intended to limit the same.

### EXAMPLES

### Example 1

Synthesis of Nucleoside Phosphoramidites

The following compounds, including amidites and their intermediates were prepared as described in U.S. Pat. No. 6,426,220 and published PCT WO 02/36743; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N4-benzoyl-5-methylcytidine penultimate intermediate for 5-methyl dC amidite, [5′-O-(4,4′-Dimethoxytriphenylmethyl)-2′-deoxy-N⁴-benzoyl-5-methylcytidin-3′-O-yl]-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, [5′-O-(4,4′-Dimethoxytriphenylmethyl)-2′-O-(2-methoxyethyl)-5-methyluridin-3′-O-yl]-2-cyanoethyl-N,N-diisopropylphosphoramidite (MOE T amidite), 5′-O-Dimethoxytrityl-2′-O-(2-methoxyethyl)-5-methylcytidine intermediate, 5′-O-dimethoxytrityl-2′-O-(2-methoxyethyl)-N⁴-benzoyl-5-methyl-cytidine penultimate intermediate, [5′-O-(4,4′-Dimethoxytriphenylmethyl)-2′-O-(2-methoxyethyl)-N⁴-benzoyl-5-methylcytidin-3′-O-yl]-2-cyanoethyl-N,N-diisopropylphosphoramidite (MOE 5-Me-C amidite), [5′-O-(4,4′-Dimethoxytriphenylmethyl)-2′-O-(2-methoxyethyl)-N⁶-benzoyladenosin-3′-O-yl]-2-cyanoethyl-N,N-diisopropylphosphoramidite (MOE A amdite), [5′-O-(4,4′-Dimethoxytriphenylmethyl)-2′-O-(2-methoxyethyl)-N⁴-isobutyrylguanosin-3′-O-yl]-2-cyanoethyl-N,N-diisopropylphosphoramidite (MOE G amidite), 2′-O-(Aminooxyethyl) nucleoside amidites and 2′-O-(dimethylaminooxyethyl) nucleoside amidites, 2′-(Dimethylaminooxyethoxy) nucleoside amidites, 5′-O-tert-Butyldiphenylsilyl-O²-2′-anhydro-5-methyluridine, 5′-O-tert-Butyldiphenylsilyl-2′-O-(2-hydroxyethyl)-5-methyluridine, 2′-O-([2-phthalimidoxy)ethyl]-5′-t-butyldiphenylsilyl-5-methyluridine 5′-O-tert-butyldiphenylsilyl-2′-O-[(2-formadoximinooxy)ethyl]-5-methyluridine, 5′-O-tert-Butyldiphenylsilyl-2′-O-[N,N dimethylaminooxyethyl]-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-[(2-cyanoethyl)-N,N-diisopropylphosphoramidite], 2′-(Aminooxyethoxy) nucleoside amidites, N2-isobutyryl-6-O-diphenylcarbamoyl-2′-O-(2-ethylacetyl)-5′-o-(4,4′-dimethoxytrityl)guanosine-3′-[(2-cyanoethyl)-N,N-diisopropylphosphoramidite], 2′-dimethylaminoethoxyethoxy (2′-DMAEOE) nucleoside amidites, 2′-O-[2(2-N,N-dimethylaminoethoxy)ethyl]-5-methyl uridine, 5′-O-dimethoxytrityl-2′-O-[2(2-N,N-dimethylaminoethoxy)-ethyl)]-5-methyl uridine and 5′-O-Dimethoxytrityl-2′-O-[2(2-N,N-dimethylaminoethoxy)-ethyl)]-5-methyl uridine-3′-O-(cyanoethyl-N,N-diisopropyl)phosphoramidite.

### Example 2

Oligonucleotide and Oligonucleoside Synthesis

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and ′preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄OAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference. 3′-Deoxy-3′-methylene phosphonate oligonucleotides are prepared as described in U.S. Pat. Nos. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 3

RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethylhydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand,. 1990, 44, 639-641; Reddy, M. P., et al., Tetrahedron Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA antisense compounds (RNA oligonucleotides) of the present invention can be synthesized by the methods herein or purchased from Dharmacon Research, Inc (Lafayette, Colo.). Once synthesized, complementary RNA antisense compounds can then be annealed by methods known in the art to form double stranded (duplexed) antisense compounds. For example, duplexes can be formed by combining 30 μl of each of the complementary strands of RNA oligonucleotides (50 uM RNA oligonucleotide solution) and 15 μl of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense compounds can be used in kits, assays, screens, or other methods to investigate the role of a target nucleic acid.

### Example 4

Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

[2′-O-Me]—[2′-deoxy]—[2′-Me] Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 394, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

[2′-O-(2-Methoxyethyl)]—[2′-deoxy]—[2′-O-(Methoxyethyl)] Chimeric Phosphorothioate Oligonucleotides

[2′-O-(2-methoxyethyl)]-[2′-deoxy]-[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

[2′-O-(2-Methoxyethyl)Phosphodiester]—[2′-deoxy Phosphorothioate]—[2′-O-(2-Methoxyethyl) Phosphodiester] Chimeric Oligonucleotides

[2′-O-(2-methoxyethyl phosphodiester]-[2′-deoxy phosphorothioate]-[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### Example 5

Design and Screening of Duplexed Antisense Compounds Targeting Interleukin 18

In accordance with the present invention, a series of nucleic acid duplexes comprising the antisense compounds of the present invention and their complements can be designed to target Interleukin 18. The nucleobase sequence of the antisense strand of the duplex comprises at least a portion of an oligonucleotide in Table 1. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG and having a two-nucleobase overhang of deoxythymidine(dT) would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense compounds are evaluated for their ability to modulate Interleukin 18 expression.

When cells reached 80% confluency, they are treated with duplexed antisense compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired duplex antisense compound at a final concentration of 200 nM. After 5 hours of treatment, the medium is replaced with fresh medium. Cells are harvested 16 hours after treatment, at which time RNA is isolated and target reduction measured by RT-PCR.

### Example 6

Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32+/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 7

Oligonucleotide Synthesis—96 Well Plate Format

Oligonucleotides were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 8

Oligonucleotide Analysis—96-Well Plate Format

The concentration of oligonucleotide in each well was assessed by dilution of samples and UV absorption spectroscopy. The full-length integrity of the individual products was evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 9

Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #353872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

NHDF Cells:

Human neonatal dermal fibroblast (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

Treatment with Antisense Compounds:

When cells reached 65-75% confluency, they were treated with oligonucleotide. For cells grown in 96-well plates, wells were washed once with 100 μL OPTI-MEM™-1 reduced-serum medium (Invitrogen Corporation, Carlsbad, Calif.) and then treated with 130 μL of OPTI-MEM™-1 containing 3.75 μg/mL LIPOFECTIN™ (Invitrogen Corporation, Carlsbad, Calif.) and the desired concentration of oligonucleotide. Cells are treated and data are obtained in triplicate. After 4-7 hours of treatment at 37° C., the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO: 1) which is targeted to human H-ras, or ISIS 18078, (GTGCGCGCGAGCCCGAAATC, SEQ ID NO: 2) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA, SEQ ID NO: 3, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf(for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM.

### Example 10

Analysis of Oligonucleotide Inhibition of Interleukin 18 Expression

Antisense modulation of Interleukin 18 expression can be assayed in a variety of ways known in the art. For example, Interleukin 18 mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. The preferred method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of Interleukin 18 can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to Interleukin 18 can be identified and obtained from a variety of sources, such as the MSRS catalog of antibodies (Aerie Corporation, Birmingham, Mich.), or can be prepared via conventional monoclonal or polyclonal antibody generation methods well known in the art.

### Example 11

Design of Phenotypic Assays and In Vivo Studies for the Use of Interleukin 18 Inhibitors

Phenotypic Assays

Once Interleukin 18 inhibitors have been identified by the methods disclosed herein, the compounds are further investigated in one or more phenotypic assays, each having measurable endpoints predictive of efficacy in the treatment of a particular disease state or condition. Phenotypic assays, kits and reagents for their use are well known to those skilled in the art and are herein used to investigate the role and/or association of Interleukin 18 in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with Interleukin 18 inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the geneotype of the cell (measurement of the expression of one or more of the genes of the cell) after treatment is also used as an indicator of the efficacy or potency of the Interleukin 18 inhibitors. Hallmark genes, or those genes suspected to be associated with a specific disease state, condition, or phenotype, are measured in both treated and untreated cells.

In Vivo Studies

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study. To account for the psychological effects of receiving treatments, volunteers are randomly given placebo or Interleukin 18 inhibitor. Furthermore, to prevent the doctors from being biased in treatments, they are not informed as to whether the medication they are administering is a Interleukin 18 inhibitor or a placebo. Using this randomization approach, each volunteer has the same chance of being given either the new treatment or the placebo.

Volunteers receive either the Interleukin 18 inhibitor or placebo for eight week period with biological parameters associated with the indicated disease state or condition being measured at the beginning (baseline measurements before any treatment), end (after the final treatment), and at regular intervals during the study period. Such measurements include the levels of nucleic acid molecules encoding Interleukin 18 or Interleukin 18 protein levels in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are healthy adults (age 18 to 65 years) and roughly an equal number of males and females participate in the study. Volunteers with certain characteristics are equally distributed for placebo and Interleukin 18 inhibitor treatment. In general, the volunteers treated with placebo have little or no response to treatment, whereas the volunteers treated with the Interleukin 18 inhibitor show positive trends in their disease state or condition index at the conclusion of the study.

### Example 12

RNA Isolation

Poly(A)+ mRNA Isolation

Poly(A)+ mRNA was isolated according to Miura et al., (Clin. Chem., 1996, 42, 1758-1764). Other methods for poly(A)+ mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

Total RNA Isolation

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 13

Real-Time Quantitative PCR Analysis of Interleukin 18 mRNA Levels

Quantitation of Interleukin 18 mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5×PCR buffer minus MgCl₂, 6.6 nM MgCl₂, 375 μM each of DATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

Probes and primers to human Interleukin 18 were designed to hybridize to a human Interleukin 18 sequence, using published sequence information (a genomic sequence of human Interleukin 18 represented by the complement of residues 16084362-16108226 of GenBank accession number NT_{—}009151.5, incorporated herein as SEQ ID NO: 4). For human Interleukin 18 the PCR primers were: forward primer: AAGGAATTGTCTCCCAGTGCAT (SEQ ID NO: 5) reverse primer: CAGGTGGCAGCCGCTTTA (SEQ ID NO: 6) and the PCR probe was: FAM-TTGCCCTCCTGGCTGCCAACTC-TAMRA (SEQ ID NO: 7) where FAM is the fluorescent dye and TAMRA is the quencher dye. For human GAPDH the PCR primers were: forward primer: GAAGGTGAAGGTCGGAGTC(SEQ ID NO:8) reverse primer: GAAGATGGTGATGGGATTTC (SEQ ID NO:9) and the PCR probe was: 5′ JOE-CAAGCTTCCCGTTCTCAGCC-TAMRA 3′ (SEQ ID NO: 10) where JOE is the fluorescent reporter dye and TAMRA is the quencher dye.

### Example 14

Northern Blot Analysis of Interleukin 18 mRNA Levels

Eighteen hours after antisense treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human Interleukin 18, a human Interleukin 18 specific probe was prepared by PCR using the forward primer AAGGAATTGTCTCCCAGTGCAT (SEQ ID NO: 5) and the reverse primer CAGGTGGCAGCCGCTTTA (SEQ ID NO: 6). To normalize for variations in loading and transfer efficiency membranes were stripped and probed for human glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 15

Antisense Inhibition of Human Interleukin 18 Expression by Chimeric Phosphorothioate Oligonucleotides having 2′-MOE Wings and a Deoxy Gap

In accordance with the present invention, a series of antisense compounds were designed to target different regions of the human Interleukin 18 RNA, using published sequences (a genomic sequence of human Interleukin 18 represented by the complement of residues 16084362-16108226 of GenBank accession number NT_{—}009151.5, incorporated herein as SEQ ID NO: 4; and GenBank accession number D49950.1, incorporated herein as SEQ ID NO: 11). The compounds are shown in Table 1. “Target Site” indicates the first (5′-most) nucleotide number on the particular target sequence to which the compound binds. All compounds in Table 1 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human Interleukin 18 mRNA levels by quantitative real-time PCR as described in other examples herein. Data are averages from three experiments in which T-24 cells were treated with the oligonucleotides of the present invention. The positive control for each datapoint is identified in the table by sequence ID number. If present, “N.D.” indicates “no data”.

TABLE 1
Inhibition of human Interleukin 18 mRNA levels by chimeric
phosphorothioate oligonucleotides having 2′-MOE wings and a
deoxy gap

| | | | | | | | |
|---|---|---|---|---|---|---|---|
| | | TARGET | | | | | CONTROL |
| | | SEQ ID | TARGET | | | SEQ ID | SEQ ID |
| ISIS # | REGION | NO | SITE | SEQUENCE | % INHIB | NO | NO |

| | | | | | | | |
|---|---|---|---|---|---|---|---|
| 163744 | Coding | 11 | 387 | tatctctacagtcagaatca | 35 | 13 | 2 |
| 163745 | Coding | 4 | 10534 | caattgtcttctactggttc | 58 | 14 | 2 |
| 163746 | Coding | 4 | 15435 | aatttagattcaagcttgcc | 0 | 15 | 2 |
| 163747 | Coding | 4 | 21856 | atgtcctgggacacttctct | 71 | 16 | 2 |
| 163748 | 5′UTR | 4 | 1600 | gccgctttagcagccagagt | 7 | 17 | 2 |
| 163749 | 5′UTR | 4 | 1612 | cagcaggtggcagccgcttt | 15 | 18 | 2 |
| 163750 | Coding | 11 | 265 | tctgattccaggttttcatc | 34 | 19 | 2 |
| 163751 | Coding | 4 | 16909 | gctatctttatacatactta | 60 | 20 | 2 |
| 163752 | 5′UTR | 4 | 1544 | tccttgctgactgtccaggc | 20 | 21 | 2 |
| 163753 | Coding | 4 | 21914 | tttcacaagctagaaagtat | 41 | 22 | 2 |
| 163754 | Coding | 4 | 10537 | atgcaattgtcttctactgg | 58 | 23 | 2 |
| 163755 | Coding | 11 | 249 | catcatcttcagctataaag | 7 | 24 | 2 |
| 163756 | 5′UTR | 4 | 1637 | cctcttcccgaagctgtgta | 87 | 25 | |
| 163757 | Coding | 4 | 16991 | aggaaataattttgttctca | 12 | 26 | 2 |
| 163758 | Coding | 4 | 16919 | ctctaggctggctatcttta | 41 | 27 | 2 |
| 163759 | Coding | 4 | 16960 | tgaaattttctcacacttca | 49 | 28 | 2 |
| 163760 | Start | 11 | 163 | gccatctttattcctgcgac | 1 | 29 | 2 |
| | Codon | | | | | | |
| 163761 | 5′UTR | 4 | 1552 | gagacaattccttgctgact | 74 | 30 | |
| 163762 | Coding | 4 | 267 | aatctgattccaggttttca | 39 | 31 | 2 |
| 163763 | Coding | 4 | 21868 | catcttattatcatgtcctg | 37 | 32 | 2 |
| 163764 | 5′UTR | 4 | 1654 | aggtctgaggttcctttcct | 37 | 33 | 2 |
| 163765 | Start | 4 | 10527 | cttctactggttcagcagcc | 58 | 34 | 2 |
| | Codon | | | | | | |
| 163766 | Coding | 4 | 16934 | tagttacagccatacctcta | 32 | 35 | 2 |
| 163767 | Coding | 11 | 251 | ttcatcatcttcagctataa | 36 | 36 | 2 |
| 163768 | Coding | 4 | 21899 | agtatccttcgtatgatgaa | 42 | 37 | 2 |
| 163769 | Start | 4 | 162 | ccatctttattcctgcgaca | 34 | 38 | 2 |
| | Codon | | | | | | |
| 163770 | 5′UTR | 4 | 1678 | tgttgcgagaggaagcgatc | 20 | 39 | 2 |
| 163771 | 5′UTR | 4 | 1618 | agactgcagcaggtggcagc | 53 | 40 | 2 |
| 163772 | Coding | 4 | 21907 | agctagaaagtatccttcgt | 68 | 41 | 2 |
| 163773 | Coding | 4 | 21873 | aattgcatcttattatcatg | 36 | 42 | 2 |
| 163774 | Coding | 4 | 15510 | gtcatatcttcaaatagagg | 11 | 43 | 2 |
| 163775 | 5′UTR | 4 | 1572 | aggagggcaaaatgcactgg | 36 | 44 | 2 |
| 163776 | Coding | 4 | 15486 | tttccttggtcaatgaagag | 0 | 45 | 2 |
| 163777 | Coding | 4 | 21995 | cgttttgaacagtgaacatt | 63 | 46 | 2 |
| 163778 | 5′UTR | 4 | 1692 | tgcgacaaatagtttgttgc | 58 | 47 | 2 |
| 163779 | Coding | 4 | 21866 | tcttattatcatgtcctggg | 51 | 48 | 2 |
| 163780 | 5′UTR | 4 | 1687 | caaatagtttgttgcgagag | 0 | 49 | 2 |
| 204541 | Coding | 4 | 10547 | cacaaagttgatgcaattgt | 80 | 50 | 2 |
| 204542 | Coding | 4 | 10553 | cattgccacaaagttgatgc | 82 | 51 | 2 |
| 204543 | Coding | 4 | 10563 | caataaatttcattgccaca | 68 | 52 | 2 |
| 204544 | Coding | 4 | 10583 | tataaagtaaagcgtattgt | 49 | 53 | 2 |
| 204545 | Coding | 4 | 15425 | caagcttgccaaagtaatct | 90 | 54 | 2 |
| 204546 | Coding | 4 | 15445 | tatgactgataatttagatt | 52 | 55 | 2 |
| 204547 | Coding | 4 | 15452 | aatttcttatgactgataat | 39 | 56 | 2 |
| 204548 | Coding | 4 | 15457 | attcaaatttcttatgactg | 43 | 57 | 2 |
| 204549 | Coding | 4 | 15465 | acttggtcattcaaatttct | 87 | 58 | 2 |
| 204550 | Coding | 4 | 15474 | atgaagagaacttggtcatt | 84 | 59 | 2 |
| 204551 | Coding | 4 | 15520 | gtcagaatcagtcatatctt | 85 | 60 | 2 |
| 204552 | Coding | 4 | 16892 | ttataataaatatggtccgg | 80 | 61 | 2 |
| 204553 | Coding | 4 | 16947 | cacttcacagagatagttac | 74 | 62 | 2 |
| 204554 | Coding | 4 | 16975 | ctcacaggagagagttgaaa | 75 | 63 | 2 |
| 204555 | Coding | 11 | 528 | gattcatttccttaaaggaa | 61 | 64 | 2 |
| 204556 | Coding | 4 | 21802 | gttatcaggaggattcattt | 84 | 65 | 2 |
| 204557 | Coding | 4 | 21813 | gtatccttgatgttatcagg | 86 | 66 | 2 |
| 204558 | Coding | 4 | 21834 | aagaatatgatgtcactttt | 68 | 67 | 2 |
| 204559 | Coding | 4 | 21837 | tgaaagaatatgatgtcact | 78 | 68 | 2 |
| 204560 | Coding | 4 | 21843 | cttctctgaaagaatatgat | 74 | 69 | 2 |
| 204561 | Coding | 4 | 21845 | cacttctctgaaagaatatg | 65 | 70 | 2 |
| 204562 | Coding | 4 | 21886 | tgatgaagattcaaattgca | 64 | 71 | 2 |
| 204563 | Coding | 4 | 21889 | gtatgatgaagattcaaatt | 79 | 72 | 2 |
| 204564 | Stop | 4 | 22005 | agctagtcttcgttttgaac | 88 | 73 | 2 |
| | Codon | | | | | | |
| 204565 | Stop | 4 | 22013 | attttaatagctagtcttcg | 79 | 74 | 2 |
| | Codon | | | | | | |
| 204566 | 5′UTR | 4 | 1533 | tgtccaggcagtcgccaggg | 74 | 75 | 2 |
| 204567 | 5′UTR | 4 | 1539 | gctgactgtccaggcagtcg | 70 | 76 | 2 |
| 204568 | Intron | 4 | 5854 | tgttcattgaatttggcaat | 56 | 77 | 2 |
| 204569 | Intron | 4 | 7001 | aatacatcccttagcacttg | 47 | 78 | 2 |
| 204570 | Intron: | 4 | 10507 | atctttattcctaatgaaag | 0 | 79 | 2 |
| | exon | | | | | | |
| | junction | | | | | | |
| 204571 | Exon: | 4 | 10594 | ttagccttacctataaagta | 15 | 80 | 2 |
| | intron | | | | | | |
| | junction | | | | | | |
| 204572 | Exon: | 4 | 17006 | tcagtcttaccttaaaggaa | 41 | 81 | 2 |
| | intron | | | | | | |
| | junction | | | | | | |
| 204573 | Intron | 4 | 17503 | catcaactggaagctgaagc | 80 | 82 | 2 |
| 204574 | Intron | 4 | 20355 | ctaagttgatgaaagtataa | 0 | 83 | 2 |
| 204575 | Intron: | 4 | 21791 | gattcatttcctatagagaa | 39 | 84 | 2 |
| | exon | | | | | | |
| | junction | | | | | | |

As shown in Table 1, SEQ ID NOs: 14, 16, 20, 22, 23, 25, 27, 28, 30, 34, 37, 40, 41, 46, 47, 48, 50, 51, 52, 53, 54, 55, 57, 58, 59, 60, 61, 62, 63, 64, 65, 66, 67, 68, 69, 70, 71, 72, 73, 74, 75, 76, 77, 78, 81 and 82 demonstrated at least 40% inhibition of human Interleukin 18 expression in this assay and are therefore preferred. More preferred are SEQ ID NOs: 16, 25 and 30. The target regions to which these preferred sequences are complementary are herein referred to as “preferred target segments” and are therefore preferred for targeting by compounds of the present invention. These preferred target segments are shown in Table 2. The sequences represent the reverse complement of the preferred antisense compounds shown in Table 1. “Target site” indicates the first (5′-most) nucleotide number on the particular target nucleic acid to which the oligonucleotide binds. Also shown in Table 2 is the species in which each of the preferred target segments was found.

TABLE 2
Sequence and position of preferred target segments identified
in Interleukin 18.

| | | | | | | |
|---|---|---|---|---|---|---|
| | TARGET | | | | | |
| | SEQ ID | TARGET | | REV COMP | | SEQ ID |
| SITEID | NO | SITE | SEQUENCE | OF SEQ ID | ACTIVE IN | NO |

| | | | | | | |
|---|---|---|---|---|---|---|
| 79224 | 12 | 10534 | gaaccagtagaagacaattg | 14 | H. sapiens | 85 |
| 79226 | 12 | 21856 | agagaagtgtcccaggacat | 16 | H. sapiens | 86 |
| 79230 | 12 | 16909 | taagtatgtataaagatagc | 20 | H. sapiens | 87 |
| 79232 | 12 | 21914 | atactttctagcttgtgaaa | 22 | H. sapiens | 88 |
| 79233 | 12 | 10537 | ccagtagaagacaattgcat | 23 | H. sapiens | 89 |
| 79235 | 12 | 1637 | tacacagcttcgggaagagg | 25 | H. sapiens | 90 |
| 79237 | 12 | 16919 | taaagatagccagcctagag | 27 | H. sapiens | 91 |
| 79238 | 12 | 16960 | tgaagtgtgagaaaatttca | 28 | H. sapiens | 92 |
| 79240 | 12 | 1552 | agtcagcaaggaattgtctc | 30 | H. sapiens | 93 |
| 79244 | 12 | 10527 | ggctgctgaaccagtagaag | 34 | H. sapiens | 94 |
| 79247 | 12 | 21899 | ttcatcatacgaaggatact | 37 | H. sapiens | 95 |
| 79250 | 12 | 1618 | gctgccacctgctgcagtct | 40 | H. sapiens | 96 |
| 79251 | 12 | 21907 | acgaaggatactttctagct | 41 | H. sapiens | 97 |
| 79256 | 12 | 21995 | aatgttcactgttcaaaacg | 46 | H. sapiens | 98 |
| 79257 | 12 | 1692 | gcaacaaactatttgtcgca | 47 | H. sapiens | 99 |
| 79258 | 12 | 21866 | cccaggacatgataataaga | 48 | H. sapiens | 100 |
| 122255 | 12 | 10547 | acaattgcatcaactttgtg | 50 | H. sapiens | 101 |
| 122256 | 12 | 10553 | gcatcaactttgtggcaatg | 51 | H. sapiens | 102 |
| 122257 | 12 | 10563 | tgtggcaatgaaatttattg | 52 | H. sapiens | 103 |
| 122258 | 12 | 10583 | acaatacgctttactttata | 53 | H. sapiens | 104 |
| 122259 | 12 | 15425 | agattactttggcaagcttg | 54 | H. sapiens | 105 |
| 122260 | 12 | 15445 | aatctaaattatcagtcata | 55 | H. sapiens | 106 |
| 122262 | 12 | 15457 | cagtcataagaaatttgaat | 57 | H. sapiens | 107 |
| 122263 | 12 | 15465 | agaaatttgaatgaccaagt | 58 | H. sapiens | 108 |
| 122264 | 12 | 15474 | aatgaccaagttctcttcat | 59 | H. sapiens | 109 |
| 122265 | 12 | 15520 | aagatatgactgattctgac | 60 | H. sapiens | 110 |
| 122266 | 12 | 16892 | ccggaccatatttattataa | 61 | H. sapiens | 111 |
| 122267 | 12 | 16947 | gtaactatctctgtgaagtg | 62 | H. sapiens | 112 |
| 122268 | 12 | 16975 | tttcaactctctcctgtgag | 63 | H. sapiens | 113 |
| 122269 | 4 | 528 | ttcctttaaggaaatgaatc | 64 | H. sapiens | 114 |
| 122270 | 12 | 21802 | aaatgaatcctcctgataac | 65 | H. sapiens | 115 |
| 122271 | 12 | 21813 | cctgataacatcaaggatac | 66 | H. sapiens | 116 |
| 122272 | 12 | 21834 | aaaagtgacatcatattctt | 67 | H. sapiens | 117 |
| 122273 | 12 | 21837 | agtgacatcatattctttca | 68 | H. sapiens | 118 |
| 122274 | 12 | 21843 | atcatattctttcagagaag | 69 | H. sapiens | 119 |
| 122275 | 12 | 21845 | catattctttcagagaagtg | 70 | H. sapiens | 120 |
| 122276 | 12 | 21886 | tgcaatttgaatcttcatca | 71 | H. sapiens | 121 |
| 122277 | 12 | 21889 | aatttgaatcttcatcatac | 72 | H. sapiens | 122 |
| 122278 | 12 | 22005 | gttcaaaacgaagactagct | 73 | H. sapiens | 123 |
| 122279 | 12 | 22013 | cgaagactagctattaaaat | 74 | H. sapiens | 124 |
| 122280 | 12 | 1533 | ccctggcgactgcctggaca | 75 | H. sapiens | 125 |
| 122281 | 12 | 1539 | cgactgcctggacagtcagc | 76 | H. sapiens | 126 |
| 122282 | 12 | 5854 | attgccaaattcaatgaaca | 77 | H. sapiens | 127 |
| 122283 | 12 | 7001 | caagtgctaagggatgtatt | 78 | H. sapiens | 128 |
| 122286 | 12 | 17006 | ttcctttaaggtaagactga | 81 | H. sapiens | 129 |
| 122287 | 12 | 17503 | gcttcagcttccagttgatg | 82 | H. sapiens | 130 |

As these “preferred target segments” have been found by experimentation to be open to, and accessible for, hybridization with the antisense compounds of the present invention, one of skill in the art will recognize or be able to ascertain, using no more than routine experimentation, further embodiments of the invention that encompass other compounds that specifically hybridize to these preferred target segments and consequently inhibit the expression of Interleukin 18.

According to the present invention, antisense compounds include antisense oligomeric compounds, antisense oligonucleotides, ribozymes, external guide sequence (EGS) oligonucleotides, alternate splicers, primers, probes, and other short oligomeric compounds which hybridize to at least a portion of the target nucleic acid.

### Example 16

Western Blot Analysis of Interleukin 18 Protein Levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to Interleukin 18 is used, with a radiolabeled or fluorescently labeled secondary antibody directed against the primary antibody species. Bands are visualized using a PHOSPHORIMAGER™ (Molecular Dynamics, Sunnyvale Calif.).

What is claimed is:

1. A compound 8 to 80 nucleobases in length targeted to a nucleic acid molecule encoding Interleukin 18, wherein said compound specifically hybridizes with said nucleic acid molecule encoding Interleukin 18 (SEQ ID NO: 4) and inhibits the expression of Interleukin 18.

2. The compound of claim 1 comprising 12 to 50 nucleobases in length.

3. The compound of claim 2 comprising 15 to 30 nucleobases in length.

4. The compound of claim 1 comprising an oligonucleotide.

5. The compound of claim 4 comprising an antisense oligonucleotide.

6. The compound of claim 4 comprising a DNA oligonucleotide.

7. The compound of claim 4 comprising an RNA oligonucleotide.

8. The compound of claim 4 comprising a chimeric oligonucleotide.

9. The compound of claim 4 wherein at least a portion of said compound hybridizes with RNA to form an oligonucleotide-RNA duplex.

10. The compound of claim 1 having at least 70% complementarity with a nucleic acid molecule encoding Interleukin 18 (SEQ ID NO: 4) said compound specifically hybridizing to and inhibiting the expression of Interleukin 18.

11. The compound of claim 1 having at least 80% complementarity with a nucleic acid molecule encoding Interleukin 18 (SEQ ID NO: 4) said compound specifically hybridizing to and inhibiting the expression of Interleukin 18.

12. The compound of claim 1 having at least 90% complementarity with a nucleic acid molecule encoding Interleukin 18 (SEQ ID NO: 4) said compound specifically hybridizing to and inhibiting the expression of Interleukin 18.

13. The compound of claim 1 having at least 95% complementarity with a nucleic acid molecule encoding Interleukin 18 (SEQ ID NO: 4) said compound specifically hybridizing to and inhibiting the expression of Interleukin 18.

14. The compound of claim 1 having at least one modified internucleoside linkage, sugar moiety, or nucleobase.

15. The compound of claim 1 having at least one 2′-O-methoxyethyl sugar moiety.

16. The compound of claim 1 having at least one phosphorothioate internucleoside linkage.

17. The compound of claim 1 having at least one 5-methylcytosine.

18. A method of inhibiting the expression of Interleukin 18 in cells or tissues comprising contacting said cells or tissues with the compound of claim 1 so that expression of Interleukin 18 is inhibited.

19. A method of screening for a modulator of Interleukin 18, the method comprising the steps of:
a. contacting a preferred target segment of a nucleic acid molecule encoding Interleukin 18 with one or more candidate modulators of Interleukin 18, and
b. identifying one or more modulators of Interleukin 18 expression which modulate the expression of Interleukin 18.

20. The method of claim 19 wherein the modulator of Interleukin 18 expression comprises an oligonucleotide, an antisense oligonucleotide, a DNA oligonucleotide, an RNA oligonucleotide, an RNA oligonucleotide having at least a portion of said RNA oligonucleotide capable of hybridizing with RNA to form an oligonucleotide-RNA duplex, or a chimeric oligonucleotide.

21. A diagnostic method for identifying a disease state comprising identifying the presence of Interleukin 18 in a sample using at least one of the primers comprising SEQ ID NOs: 5 or 6, or the probe comprising SEQ ID NO: 7.

22. A kit or assay device comprising the compound of claim 1.

23. A method of treating an animal having a disease or condition associated with Interleukin 18 comprising administering to said animal a therapeutically or prophylactically effective amount of the compound of claim 1 so that expression of Interleukin 18 is inhibited.

24. The method of claim 23 wherein the disease or condition is an autoimmune disorder.
